(19) United States
(12) Patent Application Publication
(10) Pub. No.: US 2024/0208988 A1
(43) Pub. Date: Jun. 27, 2024

(54) Alstonine Derivatives and Salts Thereof

(71) Applicant: NAPO PHARMACEUTICALS, INC., San Francisco, CA (US)

(72) Inventors: Steven King, Moss Beach, CA (US); Karen Brunke, Belmont, CA (US); Elaine Elisabetsky, São Paulo (BR); Maurice M. Iwu, Silver Spring, MD (US)

(21) Appl. No.: 18/562,605

(22) PCT Filed: Jun. 14, 2022
(86) PCT No.: PCT/US2022/033451

§ 371 Date: Nov. 20, 2023

Related U.S. Application Data
(60) Provisional application No. 63/210,255, filed on Jun. 14, 2021.

(51) Int. Cl. C07D 491/22; A61K 31/4375; A61K 45/06; A61P 25/18

(52) U.S. Cl. CPC C07D 491/22; A61K 31/4375; A61K 45/06; A61P 25/18

(57) ABSTRACT

The present disclosure is concerned with alstonine, alstonine analogs, and salts thereof for the treatment of various disorders where changes in 5-HT_{2A}/5-HT_{2C} receptor signaling and/or dopamine transporter activity may be beneficial such as, for example, psychotic disorders, epilepsy, and anxiety disorders. This abstract is intended as a scanning tool for purposes of searching in the particular art and is not intended to be limiting of the present invention.

### CROSS-REFERENCE TO RELATED APPLICATIONS

This Application claims the benefit of U.S. Application No. 63/210,255, filed on Jun. 14, 2021, the contents of which are incorporated herein by reference in their entirety.

### BACKGROUND

Schizophrenia, with a 1% estimated global prevalence, is a serious highly debilitating psychiatric disorder, and the current available treatments lack adequate efficacy, particularly regarding negative and cognitive symptoms (Dolgin (2014) Nature 508: S10-S11). Considered to be the result of a complex interplay between genetic and environmental factors (Modinos et al. (2013) Schizophr. Res. 150: 356-365), schizophrenia is thought to include dopaminergic imbalance (Kuepper et al. (2012) Handb. Exp. Pharmacol., 1-26); whereas hypoactive cortical dopaminergic regions lead to negative (e.g., social interaction withdrawal and affective flattening) and cognitive (e.g., attention and memory deficits) symptoms, hyperactive mesolimbic dopaminergic pathways correlate to positive (e.g., delusions and hallucinations) symptoms. Treatment of schizophrenia and other similar psychotic disorders usually involves administration of either typical (older) agents acting via Dopamine D2 receptor (D2R) anatagonism or atypical (newer) agents, which act via D2R antagonism in addition to various other receptors (including 5HT2A, 5HT1A, 5HT2B, and D3) (Meltzer (2013) Annu. Rev. Med. 64: 393-406).

Psychiatry co-morbidity with epilepsy is common and often requires the combined use of psychotropic and antiepileptic drugs (AEDs). The risk of antipsychotic and antidepressants to induce seizures is elevated, and experience indicates that carefully administration of these drugs is necessary for a safe use in epileptic patients (Koch-Stoecker (2002) Epilepsia 43: 19-24). Although side and toxic effects can be increased by pharmacodynamic interactions among these substances (Koch-Stoecker (2002) Epilepsia 43: 19-24), the mechanism underlying the pro-convulsant properties of antipsychotic drugs are still unclear.

Anxiety is a syndrome common to many nervous disorders and in schizophrenia, antipsychotic therapy reduces anxiety concomitant with the alleviation of the psychosis (Bourin et al. (2001) Behav Brain Res 124(1): 87-95). Recently, a significant amount of attention has been given to the links between anxiety and schizophrenia (Turnbull and Bebbington (2001) Soc Psychiatry Psychiatr Epidemiol 36(5): 235-43). For example, schizophrenic patients may exhibit depression, anxiety, and fear, often hard to distinguish from primary negative symptoms of schizophrenia. It has been suggested that the anxiolytic property of certain antipsychotics is a crucial feature for ameliorating the so-called negative symptoms, which affect the quality life in some schizophrenics (Cao and Rodgers (1997) Eur J Pharmacol 335(2-3): 117-25; Sakamoto et al. (1998) Pharmacol Biochem Behav 60(4): 873-8; Huppert et al. (2001) Schizophr Res 51(2-3): 171-80). It is known that the relative efficacy on positive and negative symptoms, cognition, psychotic anxiety and depression, suicidality and quality of life varies among different antipsychotic compounds (Blin (1999) Can J Psychiatry 44(3): 235-44); it is also recognized that differences in the diverse behavioral experimental models may be indicative of diverse clinical profile (Moore (1999) Br J Psychiatr Suppl 38: 5-11).

The indole alkaloid alstonine is the major component of a plant-based remedy, traditionally used in Nigeria to treat mental illnesses (Costa-Campos et al., 1999). While decoctions prepared with alstonine-containing plants are used orally in African traditional medicine (Iwu, 1993), intraperitonial administration to mice shows significant effects within 30 min. In mice models (haloperidol catalepsy, amphetamine lethality, amphetamine- and apomorphine-induced stereotypes), alstonine shows antipsychotic properties (0.5-1.0 mg/kg ip); the profile is closer to atypical antipsychotics (clozapine and sulpiride) than to older neuroleptics (haloperidol and chlorpromazine). Nevertheless, unlike most atypical antipsychotics, in preliminary binding studies alstonine does not affect [3H]SCH23390 or [3H]Spiperone binding to cortex membranes, indicating the lack of direct interaction with D₁, D₂, and 5-HT_{2A} receptors (Costa-Campos et al., 1998). Quantitative autoradiography (QAR) analysis in distinct brain areas of mice treated with alstonine, combined with the effects of alstonine in dopamine (DA) uptake studied in mouse striatal synaptosomes, indicate that altonine modulates DA receptors by increasing DA uptake (Linck et al. (2015) Phytomedicine 22(1): 52-5). Behavioral studies (social interaction and memory deficits) show that the serotonin receptors 5HT_{2A} and 5HT_{2C} are crucial in alstonine's mechanism of action (Linck et al. (2008) Progress in Neuro-Psychopharmacology & Biological Psychiatry 32: 1449-1452; Linck et al. (2012) Progress in Neuro-Psychopharmacology & Biological Psychiatry 26: 29-33).

Despite the significant interest in indole-based alkaloids such as alstonine, the development of therapeutic regimens using such compounds has remained limited. Thus, there remains a need for compounds and compositions for treating and preventing mental illnesses such as psychotic disorders, epilepsy, and anxiety disorders. These needs and others are met by the present invention.

### SUMMARY

In accordance with the purpose(s) of the invention, as embodied and broadly described herein, the invention, in one aspect, relates to compounds and compositions for use in treating disorders where changes in Human Serotonin Receptor 2A (5-HT_{2A})/Human Serotonin Receptor 2C (5-HT_{2C}) receptor signaling and/or dopamine transporter activity may be beneficial, such as, for example, psychotic disorders (e.g., schizophrenia, schizoaffective disorder, schizophreniform disorder, a brief psychotic disorder, delusional disorder, substance-induced psychotic disorder, paraphrenia), epilepsy, and anxiety disorders (e.g., generalized anxiety disorder, panic disorder, agoraphobia, selective mutism, separation anxiety disorder, social anxiety disorder, a specific phobia, obsessive-compulsive disorder, substance-induced anxiety disorder).

Thus, disclosed are compounds having a structure represented by a formula:

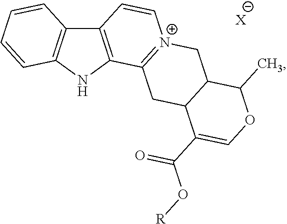

wherein X is selected from halogen, acetate, alginate, ascorbate, benzoate, carbonate, cinnamate, citrate, diphosphate, fumarate, gluconate, lactate, laurate, malate, maleate, mesylate, myristate, nitrate, palmitate, phosphate, propionate, sorbate, succinate, sulfate, tartrate, and a conjugate base of a phenolic acid; and wherein R is C1-C16 alkyl, provided that when R is methyl then X is not Cl.

Also disclosed are pharmaceutical compositions comprising an effective amount of a disclosed compound, and a pharmaceutically acceptable carrier.

Also disclosed are methods for modulating 5-HT_{2A} and/or 5-HT_{2C} receptor signaling in a subject in need thereof, the method comprising administering to the subject an effective amount of a disclosed compound.

Also disclosed are methods for modulating 5-HT_{2A} and/or 5-HT_{2C} receptor signaling in a cell, the method comprising contacting the cell with an effective amount of a disclosed compound.

Also disclosed are methods for modulating dopamine transporter activity in a subject in need thereof, the method comprising administering to the subject an effective amount of a disclosed compound.

Also disclosed are methods for modulating dopamine transporter activity in a cell, the method comprising contacting the cell with an effective amount of a disclosed compound.

Also disclosed are methods for treating a disorder in a subject in need thereof, the method comprising administering to the subject an effective amount of a disclosed compound, wherein the disorder is a psychotic disorder, an anxiety disorder, or epilepsy.

Also disclosed are kits comprising a disclosed compound, and one or more of: (a) an anti-psychotic agent; (b) an anti-anxiety agent; (c) an anti-seizure agent; (d) instructions for administering the compound in connection with treating a psychotic disorder, an anxiety disorder, or epilepsy; and (e) instructions for treating a psychotic disorder, an anxiety disorder, or epilepsy.

Also disclosed are devices comprising: (a) a compound having a structure represented by a formula:

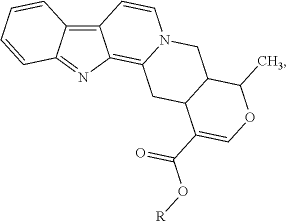

wherein R is C1-C16 alkyl, or a pharmaceutically acceptable salt thereof; (b) a microneedle array or a transdermal patch; and (c) optionally, a transdermal agent.

Also disclosed are methods for treating schizophrenia in a subject in need thereof, the method comprising transdermally administering to the subject an effective amount of a compound having a structure represented by a formula:

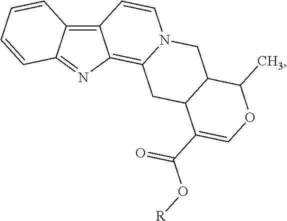

wherein R is C1-C16 alkyl, or a pharmaceutically acceptable salt thereof.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE DRAWINGS

The accompanying figures, which are incorporated in and constitute a part of this specification, illustrate several aspects and together with the description serve to explain the principles of the invention.

FIG. 1 shows a representative image illustrating the tank set up for the social interaction test.

FIG. 2A-C show representative data illustrating the effects of olanzapine (FIG. 2A), alstonine hydrochloride (FIG. 2B), and alstonine hydrobromide (FIG. 2C) on interaction time.

FIG. 3A-C show representative data illustrating the effects of olanzapine (FIG. 3A), alstonine hydrochloride (FIG. 3B), and alstonine hydrobromide (FIG. 3C) on the total distance swum in the social interaction test.

FIG. 4A-C show representative data illustrating the effects of olanzapine (FIG. 4A), alstonine hydrochloride (FIG. 4B), and alstonine hydrobromide (FIG. 4C) on the number of crossings in the social interaction test.

FIG. 5A-C show representative data illustrating the effects of olanzapine (FIG. 5A), alstonine hydrochloride (FIG. 5B), and alstonine hydrobromide (FIG. 5C) on the time spent immobile in the social interaction test.

FIG. 6 shows a representative image illustrating the tank set up for the open tank test.

FIG. 7A-C show representative data illustrating the effects of olanzapine (FIG. 7A), alstonine hydrochloride (FIG. 7B), and alstonine hydrobromide (FIG. 7C) on the total distance swum in the open tank test.

FIG. 8A-C show representative data illustrating the effects of olanzapine (FIG. 8A), alstonine hydrochloride (FIG. 8B), and alstonine hydrobromide (FIG. 8C) on the absolute turn angle in the open tank test.

FIG. 9A-C show representative data illustrating the effects of olanzapine (FIG. 9A), alstonine hydrochloride (FIG. 9B), and alstonine hydrobromide (FIG. 9C) on the clockwise rotation in the open tank test.

FIG. 10A-C show representative data illustrating the effects of olanzapine (FIG. 10A), alstonine hydrochloride (FIG. 10B), and alstonine hydrobromide (FIG. 10C) on the time spent immobile in the open tank test.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. Definitions

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein. In a further aspect, IC₅₀ refers to the half-maximal (50%) inhibitory concentration (IC) of a substance.

As used herein, “EC₅₀,” is intended to refer to the concentration of a substance (e.g., a compound or a drug) that is required for 50% agonism of a biological process, or component of a process, including a protein, subunit, organelle, ribonucleoprotein, etc. In one aspect, an EC₅₀ can refer to the concentration of a substance that is required for 50% agonism in vivo, as further defined elsewhere herein. In a further aspect, EC₅₀ refers to the concentration of agonist that provokes a response halfway between the baseline and maximum response.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; anti-cancer and anti-neoplastic agents such as kinase inhibitors, poly ADP ribose polymerase (PARP) inhibitors and other DNA damage response modifiers, epigenetic agents such as bromodomain and extra-terminal (BET) inhibitors, histone deacetylase (HDAc) inhibitors, iron chelators and other ribonucleotides reductase inhibitors, proteasome inhibitors and Nedd8-activating enzyme (NAE) inhibitors, mammalian target of rapamycin (mTOR) inhibitors, traditional cytotoxic agents such as paclitaxel, dox, irinotecan, and platinum compounds, immune checkpoint blockade agents such as cytotoxic T lymphocyte antigen-4 (CTLA-4) monoclonal antibody (mAB), programmed cell death protein 1 (PD-1)/programmed cell death-ligand 1 (PD-L1) mAB, cluster of differentiation 47 (CD47) mAB, toll-like receptor (TLR) agonists and other immune modifiers, cell therapeutics such as chimeric antigen receptor T-cell (CAR-T)/chimeric antigen receptor natural killer (CAR-NK) cells, and proteins such as interferons (IFNs), interleukins (ILs), and mAbs; anti-ALS agents such as entry inhibitors, fusion inhibitors, non-nucleoside reverse transcriptase inhibitors (NNRTIs), nucleoside reverse transcriptase inhibitors (NRTIs), nucleotide reverse transcriptase inhibitors, NCP7 inhibitors, protease inhibitors, and integrase inhibitors; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen,” or “halide,” as used herein can be used interchangeably and refer to F, Cl, Br, or I.

The terms “pseudohalide,” “pseudohalogen,” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl,” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl”, “heteroaryl”, “bicyclic heterocycle” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl,” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxyl” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogen of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{•} (or the ring formed by taking two independent occurrences of R^{•} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

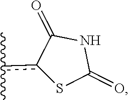

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (e.e.). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

The compounds according to this disclosure may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radio-actively labeled forms, isomers, solvates and combinations thereof. The “combinations” mentioned in this context are refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

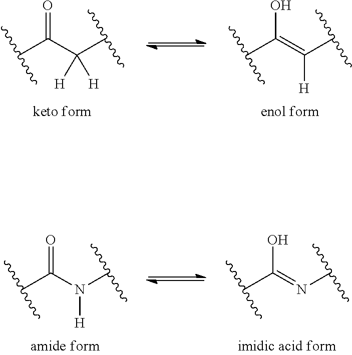 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyrazoles can exist in two tautomeric forms, N¹-unsubstituted, 3-A³ and N-unsubstituted, 5-A³ as shown below.

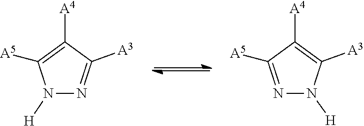

Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

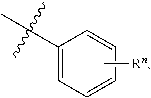

which is understood to be equivalent to a formula:

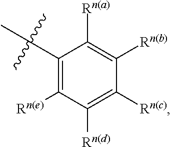

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, MA), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compounds and compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Compounds

Alstonine has been evaluated for the treatment of a variety of disorders where changes in 5-HT_{2A}A/5-HT_{2C} receptor signaling and/or dopamine transporter activity may be beneficial, including psychotic disorders (Costa-Campos et al. (1998) Pharmacology Biochemistry and Behavior 60(1): 133-141; WO 2020/072675 A1; Linck et al. (2011) Evidence-Based Complementary and Alternative Medicine, Volume 2011, Article ID 418596, 7 pages; Linck et al. (2008) Progress in Neuro-Psychopharmacology & Biological Psychiatry 32: 1449-1452; Herrmann et al. (2012) Neurochemistry International 61: 1144-1150; Linck et al. (2014) Phytomedicine 22(1): 52-5), anxiety disorders (Linck et al. (2012) Progress in Neuro-Psychopharmacology & Biological Psychiatry 26: 29-33; Costa-Campos et al. (2004) Pharmacology, Biochemistry and Behavior 77: 481-489), and epilepsy (Costa-Campos et al. (2004) Journal of Ethnopharmacology 93: 307-310). However, the utility of alstonine as a viable therapeutic strategy has remained limited. Here, alstonine and analogs thereof are converted into a salt form.

Thus, in one aspect, the invention relates to compounds useful in treating disorders where changes in 5-HT_{2A}/5-HT_{2C} receptor signaling and/or dopamine transporter activity may be beneficial such as, for example, psychotic disorders (e.g., schizophrenia, schizoaffective disorder, schizophreniform disorder, a brief psychotic disorder, delusional disorder, substance-induced psychotic disorder, paraphrenia), epilepsy, and anxiety disorders (e.g., generalized anxiety disorder, panic disorder, agoraphobia, selective mutism, separation anxiety disorder, social anxiety disorder, a specific phobia, obsessive-compulsive disorder, substance-induced anxiety disorder).

In one aspect, the compounds of the invention are useful in the treatment of psychotic disorders, as further described herein.

In one aspect, the compounds of the invention are useful in the treatment of epilepsy, as further described herein.

In one aspect, the compounds of the invention are useful in the treatment of anxiety disorders, as further described herein.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

### 1. Structure

In one aspect, disclosed are compounds having a structure represented by a formula:

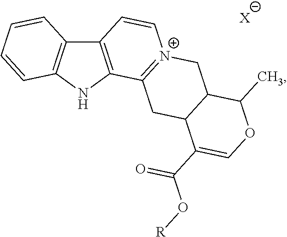

wherein X is selected from halogen, acetate, alginate, ascorbate, benzoate, carbonate, cinnamate, citrate, diphosphate, fumarate, gluconate, lactate, laurate, malate, maleate, mesylate, myristate, nitrate, palmitate, phosphate, propionate, sorbate, succinate, sulfate, tartrate, and a conjugate base of a phenolic acid; and wherein R is C1-C16 alkyl, provided that when R is methyl then X is not Cl.

In one aspect, disclosed are compounds having a structure represented by a formula:

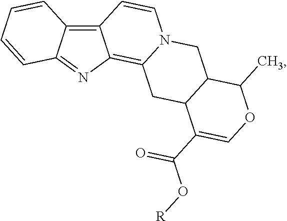

wherein R is C1-C16 alkyl, or a pharmaceutically acceptable salt thereof.

In various aspects, the compound has a structure represented by a formula:

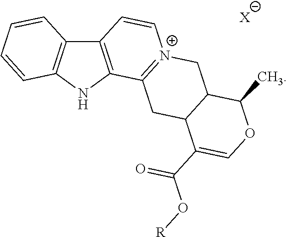

In various aspects, the compound has a structure represented by a formula:

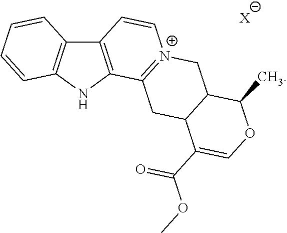

In various aspects, the compound has a structure represented by a formula:

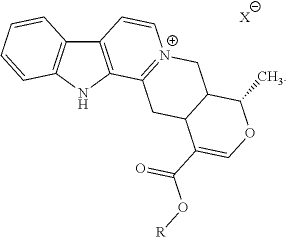

In various aspects, the compound has a structure represented by a formula:

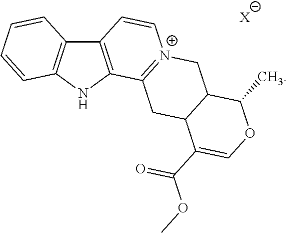

In various aspects, the compound has a structure represented by a formula:

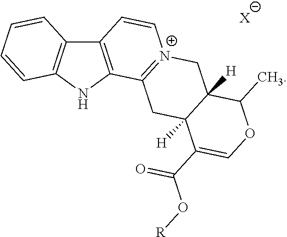

In various aspects, the compound has a structure represented by a formula:

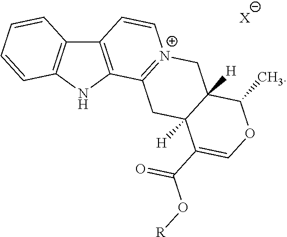

In various aspects, the compound has a structure represented by a formula:

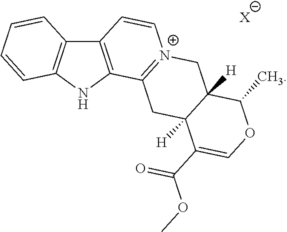

In various aspects, the compound has a structure represented by a formula:

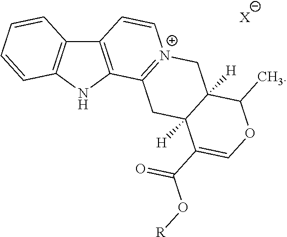

In various aspects, the compound has a structure represented by a formula:

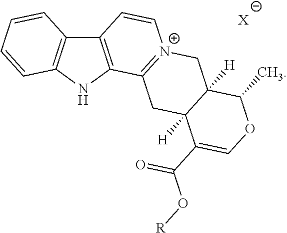

In various further aspects, X is selected from cinnamate, succinate, and trimethoxybenzoate.

In various aspects, the compound has a structure represented by a formula:

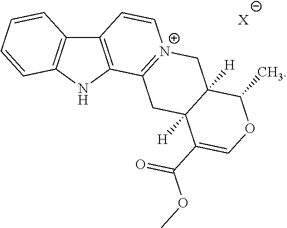

In various aspects, the compound is selected from:

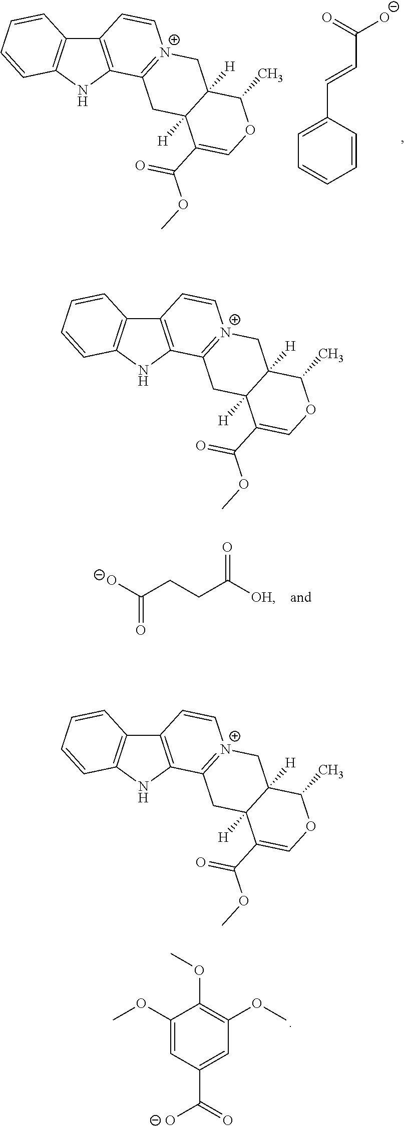

In various aspects, the compound has a structure:

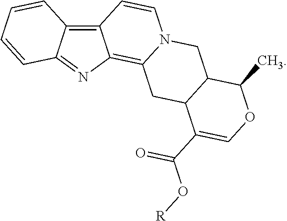

In various aspects, the compound has a structure:

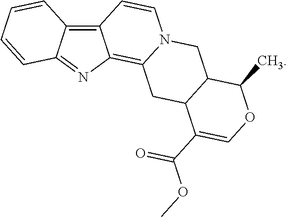 [SMILES: COC(=O)C1=CO[C@H](C)C2Cn3ccc4c5ccccc5nc-4c3CC12]

In various aspects, the compound has a structure:

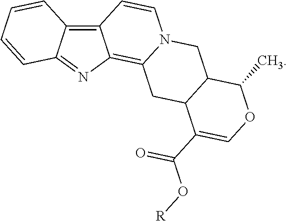

In various aspects, the compound has a structure:

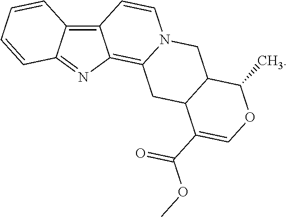 [SMILES: COC(=O)C1=CO[C@@H](C)C2Cn3ccc4c5ccccc5nc-4c3CC12]

In various aspects, the compound has a structure:

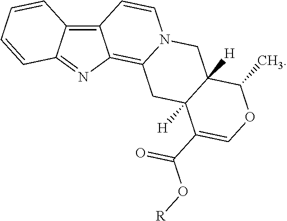

In various aspects, the compound has a structure:

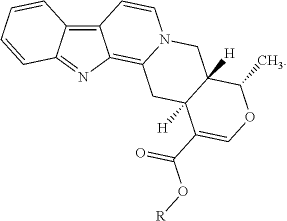

In various aspects, the compound has a structure:

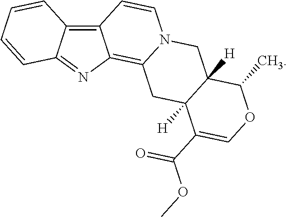 [SMILES: COC(=O)C1=CO[C@@H](C)[C@H]2Cn3ccc4c5ccccc5nc-4c3C[C@H]12]

In various aspects, the compound has a structure:

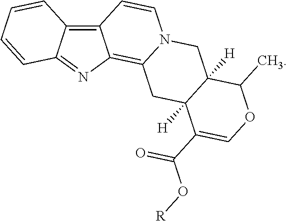

In various aspects, the compound has a structure:

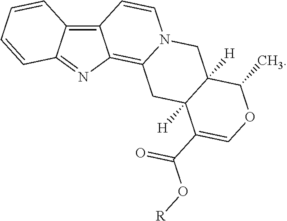

In various aspects, the compound has a structure:

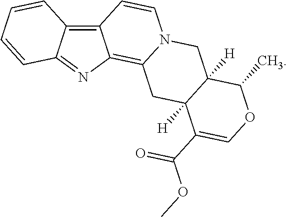 [SMILES: COC(=O)C1=CO[C@@H](C)[C@@H]2Cn3ccc4c5ccccc5nc-4c3C[C@H]12]

In various aspects, the compound is a pharmaceutically acceptable salt having a structure represented by a formula:

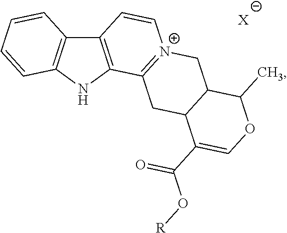

wherein X is selected from halogen, acetate, alginate, ascorbate, benzoate, carbonate, cinnamate, citrate, diphosphate, fumarate, gluconate, lactate, laurate, malate, maleate, mesylate, myristate, nitrate, palmitate, phosphate, propionate, sorbate, succinate, sulfate, tartrate, and a conjugate base of a phenolic acid, and R is as described elsewhere herein.

In various further aspects, the pharmaceutically acceptable salt has a structure represented by a formula:

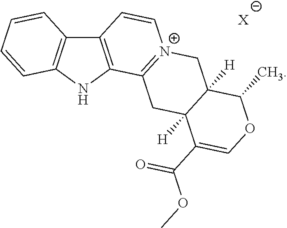

In various further aspects, the pharmaceutically acceptable salt is selected from:

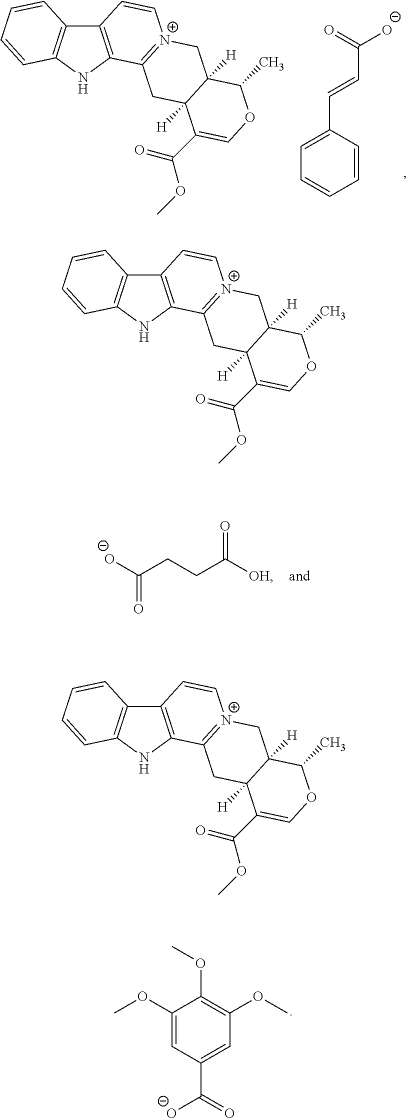

### a. X Groups

In one aspect, X is selected from halogen, acetate, alginate, ascorbate, benzoate, carbonate, cinnamate, citrate, diphosphate, fumarate, gluconate, lactate, laurate, malate, maleate, mesylate, myristate, nitrate, palmitate, phosphate, propionate, sorbate, succinate, sulfate, tartrate, and a conjugate base of a phenolic acid.

In various aspects, X is halogen. In a further aspect, X is selected from F, Br, and I. In a still further aspect, X is selected from F and Br. In yet a further aspect, X is Cl. In an even further aspect, X is F. In a still further aspect, X is Br. In yet a further aspect, X is I.

In various aspects, X is selected from acetate, alginate, ascorbate, benzoate, carbonate, cinnamate, citrate, diphosphate, fumarate, gluconate, lactate, laurate, malate, maleate, mesylate, myristate, nitrate, palmitate, phosphate, propionate, sorbate, succinate, sulfate, tartrate, and a conjugate base of a phenolic acid, and is substituted with 0-1, 0-2, or 0-3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, X is selected from acetate, alginate, ascorbate, benzoate, carbonate, cinnamate, citrate, diphosphate, fumarate, gluconate, lactate, laurate, malate, maleate, mesylate, myristate, nitrate, palmitate, phosphate, propionate, sorbate, succinate, sulfate, tartrate, and a conjugate base of a phenolic acid, and is substituted with 0-2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, X is selected from acetate, alginate, ascorbate, benzoate, carbonate, cinnamate, citrate, diphosphate, fumarate, gluconate, lactate, laurate, malate, maleate, mesylate, myristate, nitrate, palmitate, phosphate, propionate, sorbate, succinate, sulfate, tartrate, and a conjugate base of a phenolic acid, and is substituted with 0-1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, X is selected from acetate, alginate, ascorbate, benzoate, carbonate, cinnamate, citrate, diphosphate, fumarate, gluconate, lactate, laurate, malate, maleate, mesylate, myristate, nitrate, palmitate, phosphate, propionate, sorbate, succinate, sulfate, tartrate, and a conjugate base of a phenolic acid, and is monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, X is selected from acetate, alginate, ascorbate, benzoate, carbonate, cinnamate, citrate, diphosphate, fumarate, gluconate, lactate, laurate, malate, maleate, mesylate, myristate, nitrate, palmitate, phosphate, propionate, sorbate, succinate, sulfate, tartrate, and a conjugate base of a phenolic acid, and is unsubstituted.

In various aspects, X is selected from acetate, alginate, ascorbate, benzoate, carbonate, cinnamate, citrate, diphosphate, fumarate, gluconate, lactate, laurate, malate, maleate, mesylate, myristate, nitrate, palmitate, phosphate, propionate, sorbate, succinate, sulfate, and tartrate, and is unsubstituted.

In various aspects, X is benzoate substituted with 0-1, 0-2, or 0-3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, X is benzoate substituted with 0-2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, X is benzoate substituted with 0-1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, X is benzoate monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, X is unsubstituted benzoate.

In various aspects, X is benzoate substituted with 0-3 methoxy groups. In a further aspect, X is trimethoxybenzoate.

In various aspects, X is the conjugate base of a phenolic acid. In a further aspect, the phenolic acid is salicylate acid, p-hydroxybenzoic acid, p-coimaric acid, ferulic acid, syringic acid, sinapic acid, vanillic acid, caffeic acid, or 3,4-dihydroxybenzoic acid. In a still further aspect, the phenolic acid is salicylate acid.

In various aspects, X is selected from cinnamate, succinate, and trimethoxybenzoate.

### b. R Groups

In one aspect, R is C1-C16 alkyl. In a further aspect, R is C1-C12 alkyl. In a still further aspect, R is C1-C8 alkyl. In yet a further aspect, R is C1-C4 alkyl. In an even further aspect, R is methyl, ethyl, n-propyl, or isopropyl. In a still further aspect, R is methyl or ethyl. In yet a further aspect, R is ethyl. In an even further aspect, R is methyl.

In various aspects, R is C2-C16 alkyl. In a further aspect, R is C2-C12 alkyl. In a still further aspect, R is C2-C8 alkyl. In yet a further aspect, R is C2-C4 alkyl. In an even further aspect, R is ethyl, n-propyl, or isopropyl. In a still further aspect, R is ethyl.

### 2. Prophetic Compound Examples

The following compound examples are prophetic, and can be prepared using the synthesis methods described herein above and other general methods as needed as would be known to one skilled in the art. It is anticipated that the prophetic compounds would be active as antiviral agents, and such activity can be determined using the assay methods described herein below.

In one aspect, a compound can be selected from:

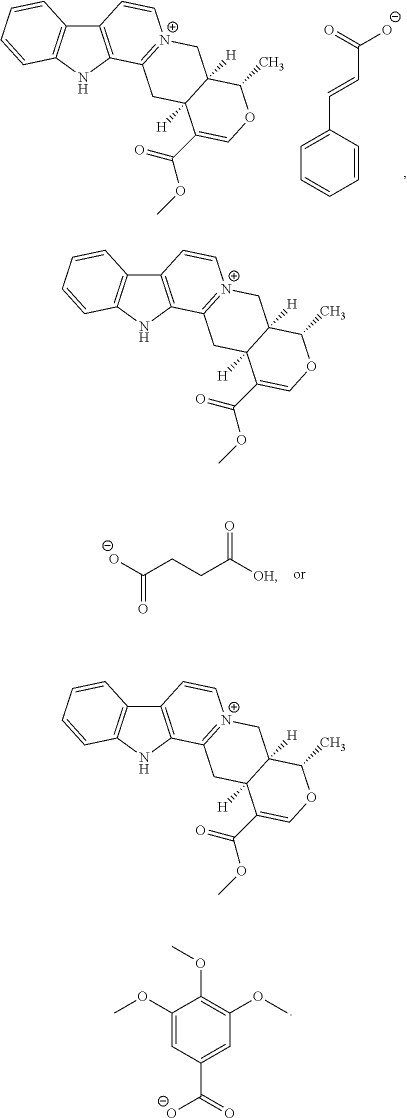

or a pharmaceutically acceptable salt thereof.

In one aspect, a compound can be selected from:

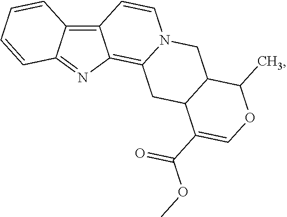 [SMILES: COC(=O)C1=COC(C)C2Cn3ccc4c5ccccc5nc-4c3CC12]

or a pharmaceutically acceptable salt thereof.

In one aspect, a compound can be selected from:

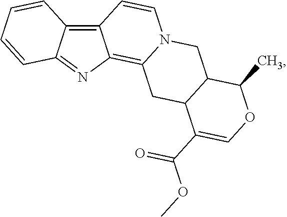 [SMILES: COC(=O)C1=CO[C@H](C)C2Cn3ccc4c5ccccc5nc-4c3CC12]

or a pharmaceutically acceptable salt thereof.

In one aspect, a compound can be selected from:

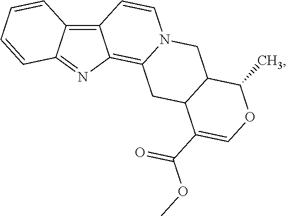 [SMILES: COC(=O)C1=CO[C@@H](C)C2Cn3ccc4c5ccccc5nc-4c3CC12]

or a pharmaceutically acceptable salt thereof.

In one aspect, a compound can be selected from:

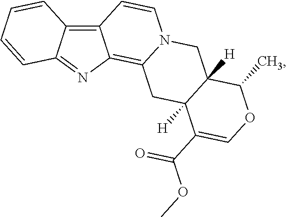 [SMILES: COC(=O)C1=CO[C@@H](C)[C@H]2Cn3ccc4c5ccccc5nc-4c3C[C@H]12]

or a pharmaceutically acceptable salt thereof.

In one aspect, a compound can be selected from:

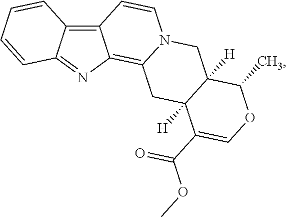 [SMILES: COC(=O)C1=CO[C@@H](C)[C@@H]2Cn3ccc4c5ccccc5nc-4c3C[C@H]12]

or a pharmaceutically acceptable salt thereof.

It is contemplated that one or more compounds can optionally be omitted from the disclosed invention.

It is understood that the disclosed compounds can be used in connection with the disclosed methods, compositions, kits, and uses.

It is understood that pharmaceutical acceptable derivatives of the disclosed compounds can be used also in connection with the disclosed methods, compositions, kits, and uses. The pharmaceutical acceptable derivatives of the compounds can include any suitable derivative, such as pharmaceutically acceptable salts as discussed below, isomers, radiolabeled analogs, tautomers, and the like.

### C. Pharmaceutical Compositions

In one aspect, disclosed are pharmaceutical compositions comprising an effective amount of a disclosed compound and a pharmaceutically acceptable carrier.

Thus, in one aspect, disclosed are pharmaceutical compositions comprising an effective amount of a compound having a structure represented by a formula:

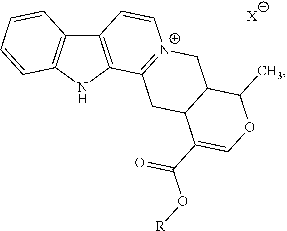

wherein X is selected from halogen, acetate, alginate, ascorbate, benzoate, carbonate, cinnamate, citrate, diphosphate, fumarate, gluconate, lactate, laurate, malate, maleate, mesylate, myristate, nitrate, palmitate, phosphate, propionate, sorbate, succinate, sulfate, tartrate, and a conjugate base of a phenolic acid; and wherein R is C1-C16 alkyl, provided that when R is methyl then X is not Cl.

In various aspects, the compounds and compositions of the invention can be administered in pharmaceutical compositions, which are formulated according to the intended method of administration. The compounds and compositions described herein can be formulated in a conventional manner using one or more physiologically acceptable carriers or excipients. For example, a pharmaceutical composition can be formulated for local or systemic administration, intravenous, topical, or oral administration.

The nature of the pharmaceutical compositions for administration is dependent on the mode of administration and can readily be determined by one of ordinary skill in the art. In various aspects, the pharmaceutical composition is sterile or sterilizable. The therapeutic compositions featured in the invention can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, water, and glycerol. The nucleic acids, polypeptides, small molecules, and other modulatory compounds featured in the invention can be administered by any standard route of administration. For example, administration can be parenteral, intravenous, subcutaneous, or oral. A modulatory compound can be formulated in various ways, according to the corresponding route of administration. For example, liquid solutions can be made for administration by drops into the ear, for injection, or for ingestion; gels or powders can be made for ingestion or topical application. Methods for making such formulations are well known and can be found in, for example, Remington's Pharmaceutical Sciences, 18th Ed., Gennaro, ed., Mack Publishing Co., Easton, PA 1990.

In various aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In various aspects, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In a further aspect, an effective amount is a therapeutically effective amount. In a still further aspect, an effective amount is a prophylactically effective amount.

In a further aspect, the pharmaceutical composition is administered to a mammal. In a still further aspect, the mammal is a human. In an even further aspect, the human is a patient.

In a further aspect, the carrier is a topical carrier. In a still further aspect, the carrier is an oral carrier. In yet a further aspect, the carrier is an injectable carrier.

In a further aspect, the composition is a topical composition. Examples of topical compositions include, but are not limited to, creams, lotions, ointments, gels, pastes, sprays, foams, powders, and solutions.

In a further aspect, the composition is an oral dosage form. Examples of oral dosage forms include, but are not limited to, tablets, capsules, lozenges, and sachets.

In a further aspect, the composition is a liquid dosage form. In a still further aspect, the composition is a solid dosage form.

In a further aspect, the composition is an injectable composition.

In a further aspect, the pharmaceutical composition is used to treat a disorder where changes in 5-HT_{2A}/5-HT_{2C} receptor signaling and/or dopamine transporter activity may be beneficial, such as, for example, psychotic disorders, anxiety disorders, and epilepsy, as further described herein.

It is understood that the disclosed compositions can be prepared from the disclosed compounds. It is also understood that the disclosed compositions can be employed in the disclosed methods of using.

### D. Methods of Making a Compound

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, as described and exemplified below. In certain specific examples, the disclosed compounds can be prepared by Routes I-III, as described and exemplified below. The following examples are provided so that the invention might be more fully understood, are illustrative only, and should not be construed as limiting.

### 1. Route I

In one aspect, compounds having a structure represented by a formula:

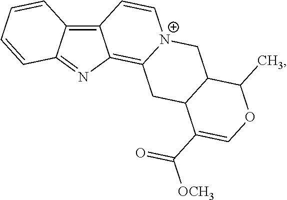 [SMILES: COC(=O)C1=COC(C)C2C[NH+]3C=CC4=c5ccccc5=NC4=C3CC12]

are commercially available, or can be prepared by methods known by those of ordinary skill in the art, such as, for example, those methods described in U.S. Pat. No. 10,323,039 B2 and 10,654,865 B2.

### 2. Route II

In one aspect, the disclosed alstonine analogs can be prepared as shown below.

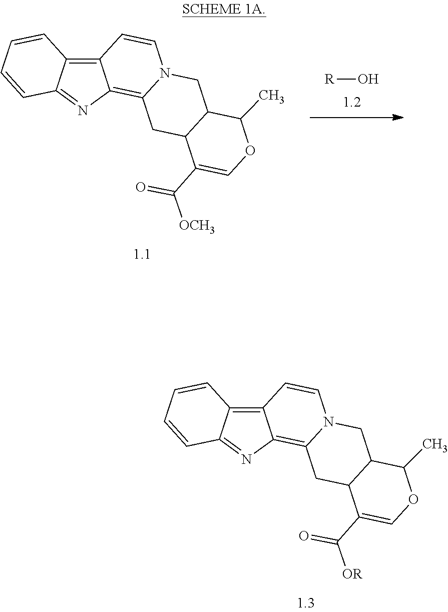

Compounds are represented in generic form, with substituents as noted in compound descriptions elsewhere herein. A more specific example is set forth below.

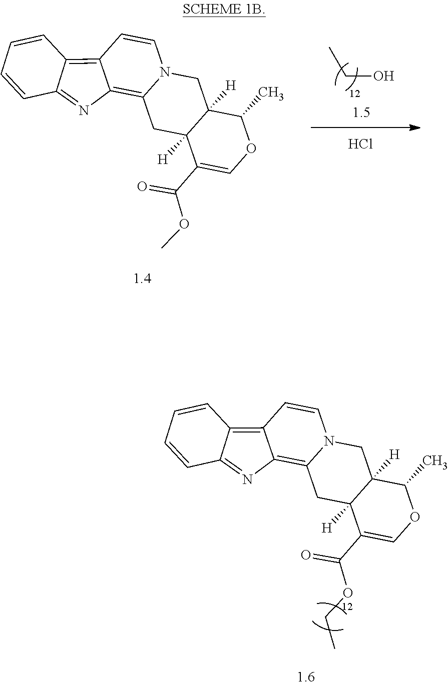

In one aspect, compounds of type 1.6 and similar compounds can be prepared according to reaction Scheme 1B above. Thus, compounds of type 1.6 can be prepared by transesterification of an appropriate methyl ester, e.g., 1.4 as shown above, using excess amounts of an appropriate alcohol, e.g., 1.5 as shown above. Appropriate methyl esters and appropriate alcohols are commercially available or prepared by methods known to one skilled in the art. The transesterification is carried out in the presence of an appropriate acid, e.g., hydrochloric acid. As can be appreciated by one skilled in the art, the above reaction provides an example of a generalized approach wherein compounds similar in structure to the specific reactants above (compounds similar to compounds of type 1.1 and 1.2) can be substituted in the reaction to provide substituted alstonine analogs similar to Formula 1.3.

### 3. Route III

In one aspect, compounds having a structure represented by a formula:

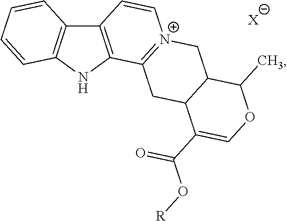

wherein X is selected from halogen, acetate, alginate, ascorbate, benzoate, carbonate, cinnamate, citrate, diphosphate, fumarate, gluconate, lactate, laurate, malate, maleate, mesylate, myristate, nitrate, palmitate, phosphate, propionate, sorbate, succinate, sulfate, tartrate, and a conjugate base of a phenolic acid; and wherein R is C1-C16 alkyl, provided that when R is methyl then X is not Cl, can be prepared by reacting a compound having a structure represented by a formula:

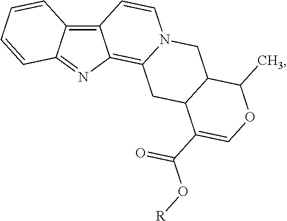

wherein R is C1-C16 alkyl, or a pharmaceutically acceptable salt thereof, and the conjugate acid of the desired anion (i.e., X⁻) in an appropriate solvent, and then precipitating out the resultant salt. Preferably, the reacting step can be carried out under anhydrous conditions to avoid hydrolysis of the ester group. The product can then optionally be purified by known methods, such as recrystallization. Alternative recrystallization methods are well known by those of ordinary skill in the art.

### E. Methods for Treating a Disorder where Changes in 5-HT_{2A}/5-HT_{2C} Receptor Signaling and/or Dopamine Transporter Activity May be Beneficial

In one aspect, disclosed are methods of treating a disorder where changes in 5-HT_{2A}/5-HT_{2C} receptor signaling and/or dopamine transporter activity may be beneficial in a subject, the method comprising the step of administering to the subject an effective amount of at least one disclosed compound, or a pharmaceutically acceptable salt thereof. In a further aspect, the disorder is a psychotic disorder, an anxiety disorder, or epilepsy.

Thus, in one aspect, disclosed are methods for treating a disorder in a subject in need thereof, the method comprising administering to the subject an effective amount of a disclosed compound, wherein the disorder is a psychotic disorder, an anxiety disorder, or epilepsy.

In one aspect, disclosed are methods for treating a disorder in a subject in need thereof, the method comprising administering to the subject an effective amount of a compound having a structure represented by a formula:

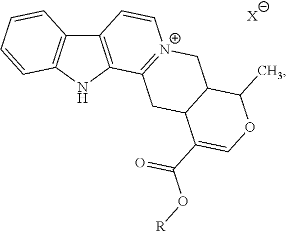

wherein X is selected from halogen, acetate, alginate, ascorbate, benzoate, carbonate, cinnamate, citrate, diphosphate, fumarate, gluconate, lactate, laurate, malate, maleate, mesylate, myristate, nitrate, palmitate, phosphate, propionate, sorbate, succinate, sulfate, tartrate, and a conjugate base of a phenolic acid; and wherein R is C1-C16 alkyl, provided that when R is methyl then X is not Cl, wherein the disorder is a psychotic disorder, an anxiety disorder, or epilepsy.

In one aspect, disclosed are methods for treating schizophrenia in a subject in need thereof, the method comprising transdermally administering to the subject an effective amount of a compound having a structure represented by a formula:

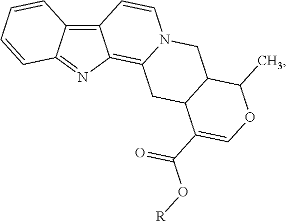

wherein R is C1-C16 alkyl, or a pharmaceutically acceptable salt thereof.

In various aspects, the compound is a pharmaceutically acceptable salt selected from:

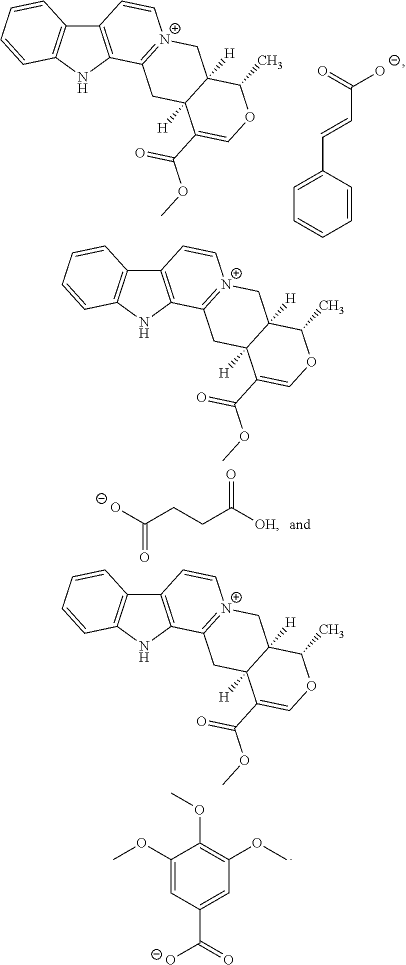

In a further aspect, the compound is formulated as a topical formulation. In a still further aspect, the topical formulation is a cream.

In a further aspect, administering is via transdermal administration. In a still further aspect, administering is via a transdermal patch. In yet a further aspect, administering is via a microneedle array.

In a further aspect, administering is via oral administration.

In a further aspect, administering is via intradermal administration.

In a further aspect, transdermally administering is via a transdermal patch or a microneedle array.

In a further aspect, the compound is administered in an amount of from about 1.0 mg/kg to about 10 mg/kg, about 1.0 mg/kg to about 8 mg/kg, about 1.0 mg/kg to about 6 mg/kg, about 1.0 mg/kg to about 4 mg/kg, about 1.0 mg/kg to about 2 mg/kg, about 2.0 mg/kg to about 10 mg/kg, about 4.0 mg/kg to about 10 mg/kg, about 6.0 mg/kg to about 10 mg/kg, about 8.0 mg/kg to about 10 mg/kg, about 2.0 mg/kg to about 8 mg/kg, about 4.0 mg/kg to about 6 mg/kg, or about 3.0 mg/kg to about 6 mg/kg.

In a further aspect, the compound is administered in an amount of from about 100 mg/day to about 500 mg/day, about 100 mg/day to about 400 mg/day, about 100 mg/day to about 300 mg/day, about 100 mg/day to about 200 mg/day, about 200 mg/day to about 500 mg/day, about 300 mg/day to about 500 mg/day, about 400 mg/day to about 500 mg/day, or about 200 mg/day to about 400 mg/day.

In a further aspect, the effective amount is a therapeutically effective amount. In a still further aspect, the effective amount is a prophylactically effective amount.

In a further aspect, the subject is a mammal. In a still further aspect, the subject is a human.

In a further aspect, the subject has been diagnosed with a need for treatment of the disorder prior to the administering step.

In a further aspect, the method further comprises the step of identifying a subject in need of treatment of the disorder.

In a further aspect, the disorder is a psychotic disorder. Examples of psychotic disorders include, but are not limited to, schizophrenia, schizoaffective disorder, schizophreniform disorder, a brief psychotic disorder, delusional disorder, substance-induced psychotic disorder, and paraphrenia. In a still further aspect, the psychotic disorder is schizophrenia.

In a further aspect, the disorder is an anxiety disorder. Examples of anxiety disorders include, but are not limited to, generalized anxiety disorder, panic disorder, agoraphobia, selective mutism, separation anxiety disorder, social anxiety disorder, a specific phobia, obsessive-compulsive disorder, or substance-induced anxiety disorder.

In a further aspect, the disorder is epilepsy.

In a further aspect, the method further comprises the step of administering an effective amount of an anti-psychotic agent. Examples of anti-pyschotic agents include, but are not limited to, aripiprazole, amisulpride, asenapine, benperidol, cariprazine, chlorpromazine, clozapine, flupentixol, fluphenazine, haloperidol, levomepromazine, lurasidone, olanzapine, paliperidone, pericyazine, pimozide, prochlorperaine, promazine, quetiapine, risperidone, sulpiride, trifluoperazine, ziprasidone, and zuclopenthixol.

In a further aspect, the compound and the anti-psychotic agent are administered sequentially. In a still further aspect, the compound and the anti-psychotic agent are administered simultaneously.

In a further aspect, the compound and the anti-psychotic agent are co-formulated. In a still further aspect, the compound and the anti-psychotic agent are co-packaged.

In a further aspect, the method further comprises the step of administering an effective amount of an anti-anxiety agent. Examples of anti-anxiety agents include, but are not limited to, amitriptyline, alprazolam, buspar, buspirex, buspirone, bustab, chlordiazepoxide, citalopram, clomipramine, clonazepam, clorazepate, desipramine, desvenlafaxine, diazepam, doxepin, duloxetine, escitalopram, fluoxetine, fluvoxamine, hydroxyzine, imipramine, klonopin, linbuspirone, lorazepam, meprobamate, midazolam, niravam, nortriptyline, oxazepam, paroxetine, pregabalin, seizalam, serax, sertraline, tranxene T-tab, venlafaxine, vilazodone, and versed.

In a further aspect, the compound and the anti-anxiety agent are administered sequentially. In a still further aspect, the compound and the anti-anxiety agent are administered simultaneously.

In a further aspect, the compound and the anti-anxiety agent are co-formulated. In a still further aspect, the compound and the anti-anxiety agent are co-packaged.

In a further aspect, the method further comprises the step of administering an effective amount of an anti-seizure agent. Examples of anti-seizure agents include, but are not limited to, acetazolamide, cannabidiol, carbamazepine, clobazam, clonazepam, diazepam, divalproex sodium, eslicarbazepine, ethosuximide, ezogabine, felbamate, gabapentin, lacosamide, lamotrigine, levetiracetam, lorazepam, methsuximide, oxcarbazepine, perampanel, phenobarbital, phenytoin, pregabalin, primidone, progabide, retigabine, rufinamide, tiagabine, topiramate, valproic acid, vigabatrin, and zonisamide.

In a further aspect, the compound and the anti-seizure agent are administered sequentially. In a still further aspect, the compound and the anti-seizure agent are administered simultaneously.

In a further aspect, the compound and the anti-seizure agent are co-formulated. In a still further aspect, the compound and the anti-seizure agent are co-packaged.

### F. Modulating 5-HT_{2A} and/or 5-HT_{2C} Activity in a Subject

In one aspect, disclosed are methods for modulating 5-HT_{2A} and/or 5-HT_{2C} receptor signaling in a subject, the method comprising the step of administering to the subject an effective amount of at least one disclosed compound, or a pharmaceutically acceptable salt thereof.

Thus, in one aspect, disclosed are methods for modulating 5-HT_{2A} and/or 5-HT_{2C} receptor signaling in a subject, the method comprising administering to the subject an effective amount of a compound having a structure represented by a formula:

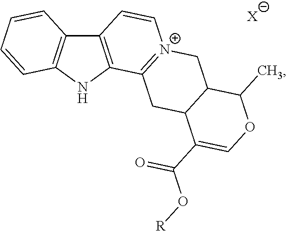

wherein X is selected from halogen, acetate, alginate, ascorbate, benzoate, carbonate, cinnamate, citrate, diphosphate, fumarate, gluconate, lactate, laurate, malate, maleate, mesylate, myristate, nitrate, palmitate, phosphate, propionate, sorbate, succinate, sulfate, tartrate, and a conjugate base of a phenolic acid; and wherein R is C1-C16 alkyl, provided that when R is methyl then X is not Cl.

In a further aspect, the subject has been diagnosed as having a disorder where changes in 5-HT_{2A} and/or 5-HT_{2C} receptor signaling may be beneficial.

In a further aspect, the subject has been diagnosed with a need for modulating 5-HT_{2A} and/or 5-HT_{2C} receptor signaling prior to the administering step.

In a further aspect, the subject has been diagnosed with a need for treating a disorder where changes in 5-HT_{2A} and/or 5-HT_{2C} receptor signaling may be beneficial prior to the administering step.

In a further aspect, the method further comprises the step of identifying a subject in need of treatment of a disorder where changes in 5-HT_{2A} and/or 5-HT_{2C} receptor signaling may be beneficial.

### G. Modulating 5-HT_{2A} and/or 5-HT_{2C} Receptor Signaling in at Least One Cell

In one aspect, disclosed are methods for modulating 5-HT_{2A} and/or 5-HT_{2C} receptor signaling in a cell, the method comprising the step of contacting the cell with an effective amount of a disclosed compound, or a pharmaceutically acceptable salt thereof.

Thus, in one aspect, disclosed are methods for modulating 5-HT_{2A} and/or 5-HT_{2C} receptor signaling in a cell, the method comprising contacting the cell with an effective amount of a compound having a structure represented by a formula:

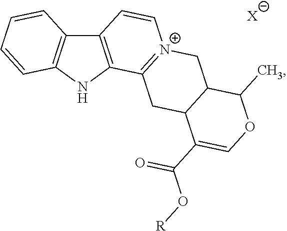

wherein X is selected from halogen, acetate, alginate, ascorbate, benzoate, carbonate, cinnamate, citrate, diphosphate, fumarate, gluconate, lactate, laurate, malate, maleate, mesylate, myristate, nitrate, palmitate, phosphate, propionate, sorbate, succinate, sulfate, tartrate, and a conjugate base of a phenolic acid; and wherein R is C1-C16 alkyl, provided that when R is methyl then X is not Cl.

In a further aspect, the cell is mammalian. In a still further aspect, the cell is human.

In a further aspect, the cell is a neuronal cell.

In a further aspect, the cell has been isolated from a human prior to the administering step.

In a further aspect, contacting is via administration to a subject. In a still further aspect, the subject has been diagnosed with a disease with a need for modulation of 5-HT_{2A} and/or 5-HT_{2C} receptor signaling prior to the administering step. In yet a further aspect, the subject has been diagnosed with a need for treatment of a disorder where changes in 5-HT_{2A} and/or 5-HT_{2C} receptor signaling may be beneficial.

### H. Modulating Dopamine Transporter Activity in a Subject

In one aspect, disclosed are methods for modulating dopamine transporter activity in a subject, the method comprising the step of administering to the subject an effective amount of at least one disclosed compound, or a pharmaceutically acceptable salt thereof.

Thus, in one aspect, disclosed are methods for modulating dopamine transporter activity in a subject, the method comprising administering to the subject an effective amount of a compound having a structure represented by a formula:

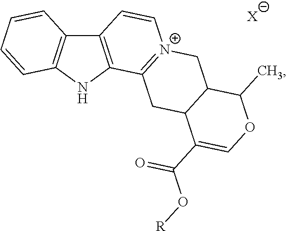

wherein X is selected from halogen, acetate, alginate, ascorbate, benzoate, carbonate, cinnamate, citrate, diphosphate, fumarate, gluconate, lactate, laurate, malate, maleate, mesylate, myristate, nitrate, palmitate, phosphate, propionate, sorbate, succinate, sulfate, tartrate, and a conjugate base of a phenolic acid; and wherein R is C1-C16 alkyl, provided that when R is methyl then X is not Cl.

In a further aspect, the subject has been diagnosed as having a disorder associated with excess dopamine signaling in distinct brain areas for which increasing the transporter activity may be beneficial.

In a further aspect, the subject has been diagnosed with a need for modulating dopamine transporter activity prior to the administering step.

In a further aspect, the subject has been diagnosed with a need for treating a disorder associated with excessive dopamine signaling in distinct brain areas for which increasing transporter activity may be beneficial prior to the administering step.

In a further aspect, the method further comprises the step of identifying a subject in need of treatment of a disorder associated with excessive signaling for which increasing dopamine transporter activity may be beneficial.

### I. Modulating Dopamine Transporter in at Least One Cell

In one aspect, disclosed are methods for modulating dopamine transporter activity in a cell, the method comprising the step of contacting the cell with an effective amount of a disclosed compound, or a pharmaceutically acceptable salt thereof.

Thus, in one aspect, disclosed are methods for modulating dopamine transporter activity in a cell, the method comprising contacting the cell with an effective amount of a compound having a structure represented by a formula:

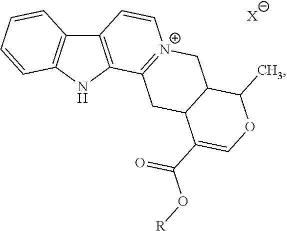

wherein X is selected from halogen, acetate, alginate, ascorbate, benzoate, carbonate, cinnamate, citrate, diphosphate, fumarate, gluconate, lactate, laurate, malate, maleate, mesylate, myristate, nitrate, palmitate, phosphate, propionate, sorbate, succinate, sulfate, tartrate, and a conjugate base of a phenolic acid; and wherein R is C1-C16 alkyl, provided that when R is methyl then X is not Cl.

In a further aspect, the cell is mammalian. In a still further aspect, the cell is human.

In a further aspect, the cell is a neuronal cell.

In a further aspect, the cell has been isolated from a human prior to the administering step.

In a further aspect, contacting is via administration to a subject. In a still further aspect, the subject has been diagnosed with a need for modulation of dopamine transporter activity prior to the administering step. In yet a further aspect, the subject has been diagnosed with a need for treatment of a disorder where changes in dopamine transporter activity may be beneficial.

### J. Devices

In one aspect, disclosed are devices comprising: (a) a compound having a structure represented by a formula:

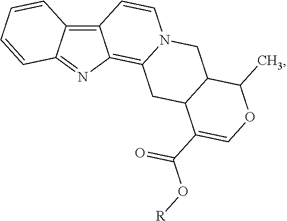

wherein R is C1-C16 alkyl, or a pharmaceutically acceptable salt thereof; (b) a microneedle array or a transdermal patch; and (c) optionally, a transdermal agent.

In various aspects, R is methyl. In various further aspect, R is C2-C16 alkyl.

In various aspects, the compound has a structure:

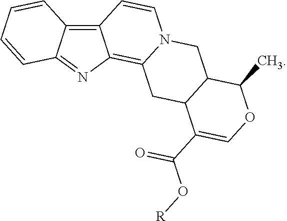

In various aspects, the compound has a structure:

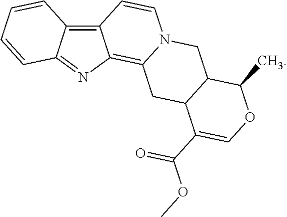 [SMILES: COC(=O)C1=CO[C@H](C)C2Cn3ccc4c5ccccc5nc-4c3CC12]

In various aspects, the compound has a structure:

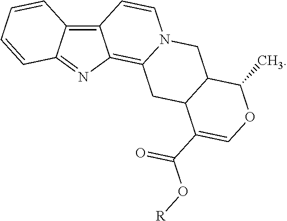

In various aspects, the compound has a structure:

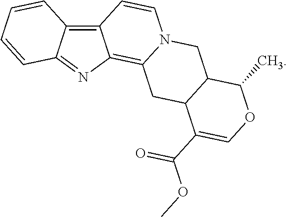 [SMILES: COC(=O)C1=CO[C@@H](C)C2Cn3ccc4c5ccccc5nc-4c3CC12]

In various aspects, the compound has a structure:

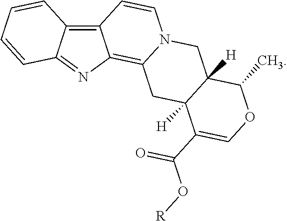

In various aspects, the compound has a structure:

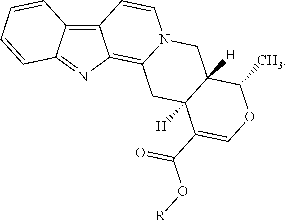

In various aspects, the compound has a structure:

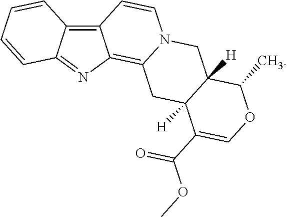 [SMILES: COC(=O)C1=CO[C@@H](C)[C@H]2Cn3ccc4c5ccccc5nc-4c3C[C@H]12]

In various aspects, the compound has a structure:

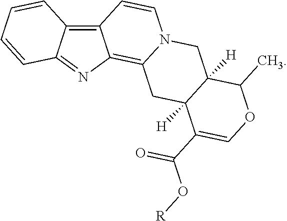

In various aspects, the compound has a structure:

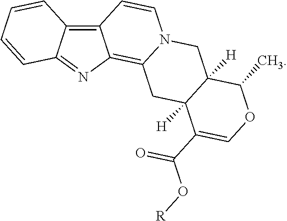

In various aspects, the compound has a structure:

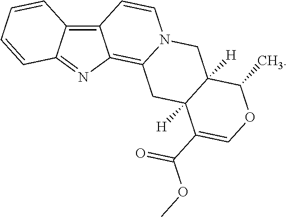 [SMILES: COC(=O)C1=CO[C@@H](C)[C@@H]2Cn3ccc4c5ccccc5nc-4c3C[C@H]12]

In various aspects, the compound is a pharmaceutically acceptable salt having a structure represented by a formula:

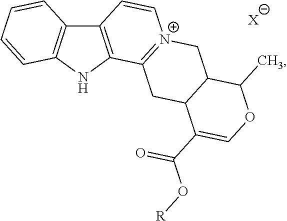

wherein X is selected from halogen, acetate, alginate, ascorbate, benzoate, carbonate, cinnamate, citrate, diphosphate, fumarate, gluconate, lactate, laurate, malate, maleate, mesylate, myristate, nitrate, palmitate, phosphate, propionate, sorbate, succinate, sulfate, tartrate, and a conjugate base of a phenolic acid, and R is as described elsewhere herein.

In various aspects, X is Cl.

In various aspects, X is selected from cinnamate, succinate, and trimethoxybenzoate.

In various further aspects, the pharmaceutically acceptable salt has a structure represented by a formula:

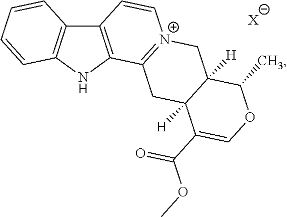

In various further aspects, the pharmaceutically acceptable salt is selected from:

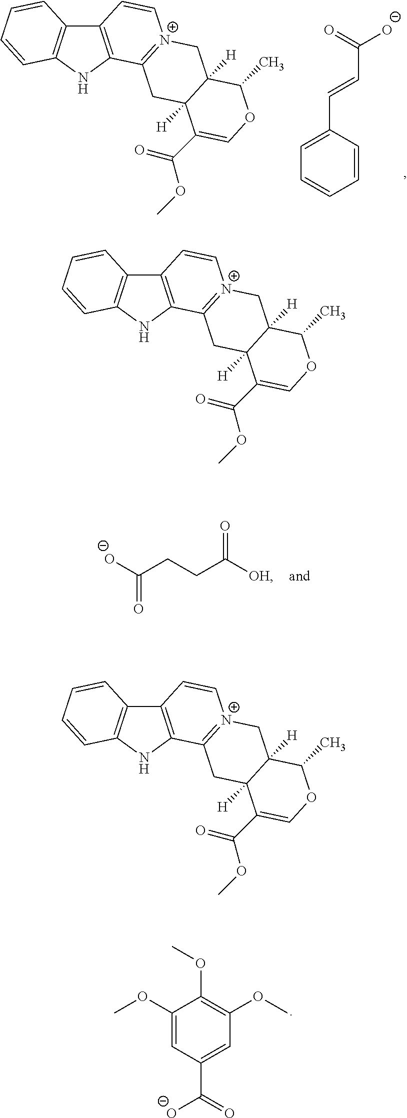

In various aspects, the device comprises the microneedle array.

In various aspects, the device comprises the transdermal patch.

In various aspects, the device comprises the transdermal agent. Examples of transdermal agents include, but are not limited to, myristates (e.g., isopropyl myristate), glycols (e.g., propylene glycol), surfactants, terpenes, azones (e.g., laurocapram), sulfoxides (e.g., dimethylsulfoxide), and pyrrolidones. In a further aspect, the transdermal agent is isopropyl myristate.

### K. Additional Methods of Using the Compounds

The compounds and pharmaceutical compositions of the invention are useful in treating or controlling disorders where changes in 5-HT_{2A} and/or 5-HT_{2C} receptor signaling, and/or dopamine transporter activity may be beneficial, such as, for example, psychotic disorders (e.g., schizophrenia, schizoaffective disorder, schizophreniform disorder, a brief psychotic disorder, delusional disorder, substance-induced psychotic disorder, paraphrenia), epilepsy, and anxiety disorders (e.g., generalized anxiety disorder, panic disorder, agoraphobia, selective mutism, separation anxiety disorder, social anxiety disorder, a specific phobia, obsessive-compulsive disorder, substance-induced anxiety disorder).

To treat or control the disorder, the compounds and pharmaceutical compositions comprising the compounds are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of a disorder where changes in 5-HT_{2A}/5-HT_{2C} receptor signaling and/or dopamine transporter activity may be beneficial, such as, for example, psychotic disorders, anxiety disorders, and epilepsy.

The compounds or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of a disorder where changes in 5-HT_{2A}/5-HT_{2C} receptor signaling and/or dopamine transporter activity may be beneficial, such as, for example, psychotic disorders, anxiety disorders, and epilepsy.

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

### 1. Use of Compounds

In one aspect, the invention relates to the use of a disclosed compound or a product of a disclosed method. In a further aspect, a use relates to the manufacture of a medicament for the treatment of a disorder where changes in 5-HT_{2A}/5-HT_{2C} receptor signaling and/or dopamine transporter activity may be beneficial (e.g., psychotic disorders, anxiety disorders, epilepsy) in a subject.

Also provided are the uses of the disclosed compounds and products. In one aspect, the invention relates to use of at least one disclosed compound; or a pharmaceutically acceptable salt, hydrate, solvate, or polymorph thereof. In a further aspect, the compound used is a product of a disclosed method of making.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, for use as a medicament.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, wherein a pharmaceutically acceptable carrier is intimately mixed with a therapeutically effective amount of the compound or the product of a disclosed method of making.

In various aspects, the use relates to a treatment of a disorder where changes in 5-HT_{2A}/5-HT_{2C} receptor signaling and/or dopamine transporter activity may be beneficial (e.g., psychotic disorders, anxiety disorders, epilepsy) in a subject. In one aspect, the use is characterized in that the subject is a human. In one aspect, the use is characterized in that the disorder is a psychotic disorder (e.g., schizophrenia, schizoaffective disorder, schizophreniform disorder, a brief psychotic disorder, delusional disorder, substance-induced psychotic disorder, paraphrenia), epilepsy, or an anxiety disorder (e.g., generalized anxiety disorder, panic disorder, agoraphobia, selective mutism, separation anxiety disorder, social anxiety disorder, a specific phobia, obsessive-compulsive disorder, substance-induced anxiety disorder).

In a further aspect, the use relates to the manufacture of a medicament for the treatment of a disorder where changes in 5-HT_{2A}/5-HT_{2C} receptor signaling and/or dopamine transporter activity may be beneficial (e.g., psychotic disorders, anxiety disorders, epilepsy).

It is understood that the disclosed uses can be employed in connection with the disclosed compounds, products of disclosed methods of making, methods, compositions, and kits. In a further aspect, the invention relates to the use of a disclosed compound or a disclosed product in the manufacture of a medicament for the disorder where changes in 5-HT_{2A}/5-HT_{2C} receptor signaling and/or dopamine transporter activity may be beneficial (e.g., psychotic disorders, anxiety disorders, epilepsy) in a mammal. In a further aspect, the disorder is a psychotic disorder (e.g., schizophrenia, schizoaffective disorder, schizophreniform disorder, a brief psychotic disorder, delusional disorder, substance-induced psychotic disorder, paraphrenia), epilepsy, or an anxiety disorder (e.g., generalized anxiety disorder, panic disorder, agoraphobia, selective mutism, separation anxiety disorder, social anxiety disorder, a specific phobia, obsessive-compulsive disorder, substance-induced anxiety disorder).

### 2. Manufacture of a Medicament

In one aspect, the invention relates to a method for the manufacture of a medicament for treating a disorder where changes in 5-HT_{2A}/5-HT_{2C} receptor signaling and/or dopamine transporter activity may be beneficial (e.g., psychotic disorders, anxiety disorders, epilepsy) in a subject having the disorder, the method comprising combining a therapeutically effective amount of a disclosed compound or product of a disclosed method with a pharmaceutically acceptable carrier or diluent.

As regards these applications, the present method includes the administration to an animal, particularly a mammal, and more particularly a human, of a therapeutically effective amount of the compound effective in the treatment of a disorder where changes in 5-HT_{2A}/5-HT_{2C} receptor signaling and/or dopamine transporter activity may be beneficial (e.g., psychotic disorders, anxiety disorders, epilepsy). The dose administered to an animal, particularly a human, in the context of the present invention should be sufficient to affect a therapeutic response in the animal over a reasonable time-frame. One skilled in the art will recognize that dosage will depend upon a variety of factors including the condition of the animal and the body weight of the animal.

The total amount of the compound of the present disclosure administered in a typical treatment is preferably between about 0.05 mg/kg and about 100 mg/kg of body weight for mice, and more preferably between 0.05 mg/kg and about 50 mg/kg of body weight for mice, and between about 100 mg/kg and about 500 mg/kg of body weight, and more preferably between 200 mg/kg and about 400 mg/kg of body weight for humans per daily dose. This total amount is typically, but not necessarily, administered as a series of smaller doses over a period of about one time per day to about three times per day for about 24 months, and preferably over a period of twice per day for about 12 months.

The size of the dose also will be determined by the route, timing and frequency of administration as well as the existence, nature and extent of any adverse side effects that might accompany the administration of the compound and the desired physiological effect. It will be appreciated by one of skill in the art that various conditions or disease states, in particular chronic conditions or disease states, may require prolonged treatment involving multiple administrations.

Thus, in one aspect, the invention relates to the manufacture of a medicament comprising combining a disclosed compound or a product of a disclosed method of making, or a pharmaceutically acceptable salt, solvate, or polymorph thereof, with a pharmaceutically acceptable carrier or diluent.

### 3. Kits

In one aspect, disclosed are kits comprising an effective amount of a disclosed compound, and one or more of: (a) an anti-psychotic agent; (b) an anti-anxiety agent; (c) an anti-seizure agent; (d) instructions for administering the compound in connection with treating a psychotic disorder, an anxiety disorder, or epilepsy; and (e) instructions for treating a psychotic disorder, an anxiety disorder, or epilepsy.

Thus, in one aspect, disclosed are kits comprising a compound having a structure represented by a formula:

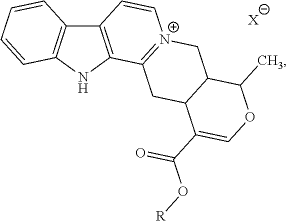

wherein X is selected from halogen, acetate, alginate, ascorbate, benzoate, carbonate, cinnamate, citrate, diphosphate, fumarate, gluconate, lactate, laurate, malate, maleate, mesylate, myristate, nitrate, palmitate, phosphate, propionate, sorbate, succinate, sulfate, tartrate, and a conjugate base of a phenolic acid; and wherein R is C1-C16 alkyl, provided that when R is methyl then X is not Cl, and one or more of: (a) an anti-psychotic agent; (b) an anti-anxiety agent; (c) an anti-seizure agent; (d) instructions for administering the compound in connection with treating a psychotic disorder, an anxiety disorder, or epilepsy; and (e) instructions for treating a psychotic disorder, an anxiety disorder, or epilepsy.

In a further aspect, the disorder is a psychotic disorder. Examples of psychotic disorders include, but are not limited to, schizophrenia, schizoaffective disorder, schizophreniform disorder, a brief psychotic disorder, delusional disorder, substance-induced psychotic disorder, and paraphrenia. In a still further aspect, the psychotic disorder is schizophrenia.

In a further aspect, the disorder is an anxiety disorder. Examples of anxiety disorders include, but are not limited to, generalized anxiety disorder, panic disorder, agoraphobia, selective mutism, separation anxiety disorder, social anxiety disorder, a specific phobia, obsessive-compulsive disorder, or substance-induced anxiety disorder.

In a further aspect, the disorder is epilepsy.

In a further aspect, the kit comprises the anti-psychotic agent. Examples of anti-pyschotic agents include, but are not limited to, aripiprazole, amisulpride, asenapine, benperidol, cariprazine, chlorpromazine, clozapine, flupentixol, fluphenazine, haloperidol, levomepromazine, lurasidone, olanzapine, paliperidone, pericyazine, pimozide, prochlorperaine, promazine, quetiapine, risperidone, sulpiride, trifluoperazine, ziprasidone, and zuclopenthixol.

In a further aspect, the compound and the anti-psychotic agent are co-formulated. In a still further aspect, the compound and the anti-psychotic agent are co-packaged.

In a further aspect, the kit comprises the anti-anxiety agent. Examples of anti-anxiety agents include, but are not limited to, amitriptyline, alprazolam, buspar, buspirex, buspirone, bustab, chlordiazepoxide, citalopram, clomipramine, clonazepam, clorazepate, desipramine, desvenlafaxine, diazepam, doxepin, duloxetine, escitalopram, fluoxetine, fluvoxamine, hydroxyzine, imipramine, klonopin, linbuspirone, lorazepam, meprobamate, midazolam, niravam, nortriptyline, oxazepam, paroxetine, pregabalin, seizalam, serax, sertraline, tranxene T-tab, venlafaxine, vilazodone, and versed.

In a further aspect, the compound and the anti-anxiety agent are co-formulated. In a still further aspect, the compound and the anti-anxiety agent are co-packaged.

In a further aspect, the kit comprises the anti-seizure agent. Examples of anti-seizure agents include, but are not limited to, acetazolamide, cannabidiol, carbamazepine, clobazam, clonazepam, diazepam, divalproex sodium, eslicarbazepine, ethosuximide, ezogabine, felbamate, gabapentin, lacosamide, lamotrigine, levetiracetam, lorazepam, methsuximide, oxcarbazepine, perampanel, phenobarbital, phenytoin, pregabalin, primidone, progabide, retigabine, rufinamide, tiagabine, topiramate, valproic acid, vigabatrin, and zonisamide.

In a further aspect, the compound and the anti-seizure agent are co-formulated. In a still further aspect, the compound and the anti-seizure agent are co-packaged.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds, products, and pharmaceutical compositions. It is also understood that the disclosed kits can be employed in connection with the disclosed methods of using.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### L. Examples

The following examples are put forth so as to provide those of ordinary skill in the art with a complete disclosure and description of how the compounds, compositions, articles, devices and/or methods claimed herein are made and evaluated, and are intended to be purely exemplary of the invention, and are not intended to limit the scope of what the inventors regard as their invention.

### 1. Isolation of Alstonine from Rind Fruits of Picralima nitida

A methanol extract of the rind fruits of Piralima nitida were subjected to column chromatography using the conditions summarized below in Table 1.

TABLE 1
Condition

| | |
|---|---|
| Mobile phase | CH₂Cl₂: Acetone |
| Stationary phase | Aluminum oxide active basic |
| Preparation of the sample | Dried extract on the stationary |
| | phase; ratio (1:1) |
| Volume collected per fraction | 50 mL |
| Weight of the extract | 350 g |

Fractions of 50 mL were collected and pooled together on the basis of their TLC profiles. After repeated chromatography with the same mobile and stationary phases to yield alstonine (350 mg). The steps were repeated until the desired quantity was obtained. A summary of the results is shown in Table 2.

TABLE 2

| | Fractions | | |
|---|---|---|---|
| Eluent | collected | Remarks | Products obtained |
| CH₂Cl₂ | 1-15 | | |
| 100% | | | |
| CH₂Cl₂- | | | |
| Acetone | | | |
| 9:1 | 16-25 | | Alstonine + other |
| | | | compounds |
| 8:2 | 26-30 | | Alstonine + other |
| | | | compounds |
| 7:3 | 31-40 | | Alstonine + other |
| | | | compounds |
| 6:4 | 41-42 | | Alstonine |
| 5:5 | 43-48 | | |
| Acetone | 48- | | |
| 100% | | | |

### 2. Preparation and Isolation of Alstonine Chloride and Alstonine Bromide

### a. Alstonine Chloride

A slurry of amberite chloride and alstonine was prepared for 48 hours. The filtrate was concentrated to dryness to yield a residue.

The resultant residue was further purified by column chromatography (stationary phase: amberlite chloride, Sephadex LH20; mobile phase: methylene chloride and methanol) to yield alstonine chloride.

### b. Alstonine Bromide

A slurry of amberite chloride and sodium bromide was prepared using water for 48 hours. The water was removed, and the prepared amberlite bromide was washed several times with methylene chloride.

A slurry of the amberlite bromide and alstonine was prepared for 48 hours using methylene chloride as the mobile phase. The filtrate was concentrated to dryness to yield a residue.

The resultant residue was further purified by column chromatography (stationary phase: Amberlite chloride, Sephadex LH20; mobile phase: methylene chloride and methanol) to yield Alstonine bromide.

### 3. Zebrafish Behavioral Assays

Zebrafish (Danio rerio) have a conserved neural architecture analogous to mammals, including cell types, neurotransmitters, and receptors (Benvenutti et al. (2021) Journal of Neuroscience Research 00: 1-16). Despite the absence of midbrain dopaminergic neurons in zebrafish and other teleost fish, populations of tyrosine hydroxylase-immunorecative cells have been identified in the diencephalon and telencephalon (Meek and Joosten (1993) Journal of Chemical Neuoranatomy 6(6): 431-446; Parker et al. (2013) Frontiers in Neural Circuites 7: 63; Rink and Guo (2004) Neuroscience 127(1): 147-154; Rink and Wullimann (2001) Brain Research 889(1-2): 316-330), with structure and function similar to birds and mammals (Matsui (2017) Development Growth and Differentiation 59(4): 219-227; Rink and Wullimann (2001) Brain Research 889(1-2): 316-330). Furthermore, glutamatergic and GABAergic neurons exert an important regulatory role in the zebrafish brain (Parker et al. (2013) Frontiers in Neural Circuites 7: 63). The presence of these neuronal systems and their organization indicate it is feasible to use zebrafish to investigate the impact of psychotropic drugs on behavior and neurochemistry (Benvenutti et al. (2021) Journal of Neuroscience Research 00: 1-16). See also Stewart et al. (2014) Trends in Neuroscience 37(5): 264-278, Patton et al. (2021) Nature Reviews 20: 611-628, and Kalueff et al. (2014) Trends in Pharmacological Sciences 36(2): 63-75, which describe zebrafish as a model organism in biomedical, pharmacogenetic, and neuropharmacological research.

MK-801 (dizocilpine) is a noncompetitive N-methyl-D-aspartate receptor (NMDAR) antagonist that mimics that hypofunction of NMDARs (Jones et al. (2011) British of Journal Pharmacology 164(4): 1162-1194). The inhibition of NMDARs in humans and animals leads to behavioral effects that resemble the full spectrum of positive, negative, and cognitive symptoms of schizophrenia (Coyle et al. (2012) Handbook of Experimental Pharmacology 213: 267-295; Hardingham and Do (2016) Nature Reviews Neuroscience 17(2): 125-134; Krystal et al. (1994) Archives of General Psychiatry 62(9): 985-995; Morris et al. (1986) Nature 319(6056): 774-776). In mice and rats, MK-801 causes hyperlocomotion, stereotypic behavior, decreased social interaction, sensorimotor gating deficits, and cognitive impairment (Jones et al. (2011) British of Journal Pharmacology 164(4): 1162-1194). In zebrafish, a few studies have shown that acute exposure to MK-801 causes hyperlocomotion (Franscescon et al. (2020) Neurochemistry International 135: 104710; Menezes et al. (2015) Zebrafish 12(2): 137-143; Tran et al. (2016) Behavioral Brain Research 296: 26-29), social deficits (Zimmermann et al. (2016) Behavioral Brain Research 311: 368-374), and cognitive impairments (Cognato et al. (2012) Neurobiology of Learning and Memory 98(4): 321-328; Franscescon et al. (2020) Neurochemistry International 135: 104710; Gaspary et al. (2018) Neurobiology of Learning and Memory 155: 249-260), with fish submitted individually to the behavioral tasks. More recently, the effects of acute exposure to MK-801 in adult zebrafish was investigated by analyzing locomotor activity, stereotypy-related behaviors, social behavior, and neurochemical parameters of oxidative status as translatable markers (Benvenutti et al. (2021) Journal of Neuroscience Research 00: 1-16).

Here, the ability of alstonine salts to reverse schizophrenia-like symptoms produced by MK-801 in zebrafish was evaluated. The atypical antipsychotic olanzapine was used as a comparison.

### a. Animals and Maintenance

Experiments were performed using male and female (50:50 ratio) short-fin wild-type zebrafish (4-month-old, 300-500 mg). Adult animals were obtained from a local commercial supplier (Delphis, RS, Brazil). The animals were maintained at Instituto de Ciências Básicas de Saúde in a light/dark cycle of 14/10 h with lights on at 7:00 a.m. for at least two weeks before tests. Fish were kept in 40-L (30×46×33 cm) unenriched glass tanks with non-chlorinated water at a maximum density of five animals per liter. Tank water satisfied the controlled conditions required for zebrafish (26±2° C.; pH 7.0±0.3; dissolved oxygen at 7.0±0.4 mg/L; total ammonia at <0.01 mg/L; total hardness at 5.8 mg/L; alkalinity at 22 mg/L CaCO₃; conductivity of 1,500-1,600 μS/cm) and was constantly filtered by mechanical, biological, and chemical filtration systems. Food was provided twice a day as commercial flake food (Poytara®, SP, Brazil) plus brine shrimp (Artemia salina). The sex of the animals was confirmed after euthanasia by dissecting and analyzing the gonads. For all experiments, no sex effects were observed, so data was pooled together. Animals were euthanized by hypothermic shock according to the AVMA Guidelines for the Euthanasia of Animals (Leary and Johnson, 2020).

### b. Chemicals

Olanzapine and MK-801 (dizocilpine) were purchased from Sigma-Aldrich (St. Louis, MO, USA). Alstonine hydrochloride and alstonine hydrobromide were prepared as described herein. MK-801 and either olanzapine or alstonine salt were dissolved in distilled water; alstonine salt stock solutions were sonicated in an ultrasonic bath. MK-801 solutions were freshly prepared, while alstonine salt stock solutions were kept refrigerated for up to five days. Exposure solutions were renovated halfway through the experiment, with a maximum of four fish exposed to the same solution.

### c. Experimental Design

Different sets of animals were used for each experiment, totaling 240 zebrafish in the study (plus 80 zebrafish serving as stimulus animals). Animals were individually exposed to water, olanzapine, or alstonine salt solutions at 2.5, 5, or 10 mg/L in 500-mL beakers containing 200-mL solution for 20 min. These concentrations were extrapolated from mice studies. Fish were then transferred to another beaker containing either water or MK-801 at 5 μM and remained there for another 20 minutes. These resulted in a full 4×2 factorial design with 8 experimental groups in each independent experiment.

The animals were allocated to the experimental groups following block randomization procedures to counterbalance for home tank, sex, and day of testing. The order for outcome assessment was also randomized and care was taken to counterbalance the test apparatuses across the experimental groups. Outcome assessors were blind to the experimental groups, as well as the experimenters responsible for taking the animal and placing it in the test apparatus.

### d. Social Interaction Test

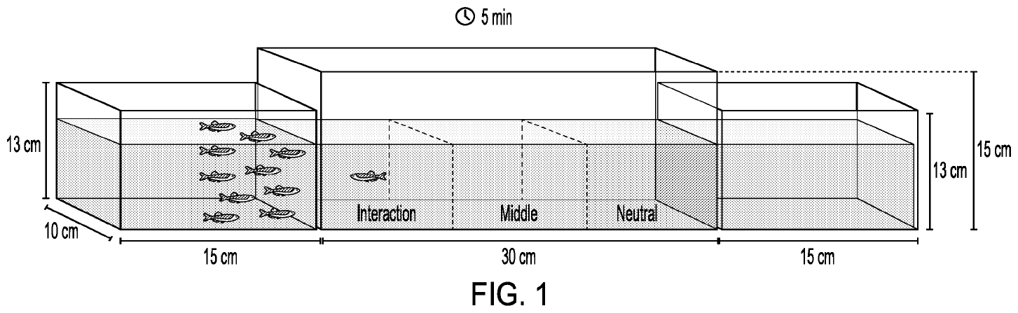

The protocol for the social interaction test in zebrafish followed the method described by Benvenutti et al. (2021) Journal of Neuroscience Research 00: 1-16. After exposure, animals were placed for 7 min in a tank (30×10×15 cm) flanked by two identical tanks (15×10×13 cm) either empty (neutral stimulus) or containing 10 unknown zebrafish (social stimulus). All three tanks were filled with water in standard conditions at a level of 10 cm. The position of the social stimulus (right or left) was counterbalanced throughout the tests. The water in the test tanks was changed between every animal. To assess social behavior, the test apparatus was virtually divided into three vertical zones (interaction, middle, and neutral). Animals were habituated to the apparatus for 2 min and then analyzed for the last 5 min. Videos were recorded from the front view and time spent in the interaction zone was quantified as a proxy for social interaction. Additionally, total distance traveled, number of crossings between the vertical zones of the tank and immobility time were quantified as secondary locomotor parameters. All outcomes were automatically scored using ANY-Maze software (Stoelting Co., Wood Dale, IL, USA). See FIG. 1.

### e. Open Tank Test

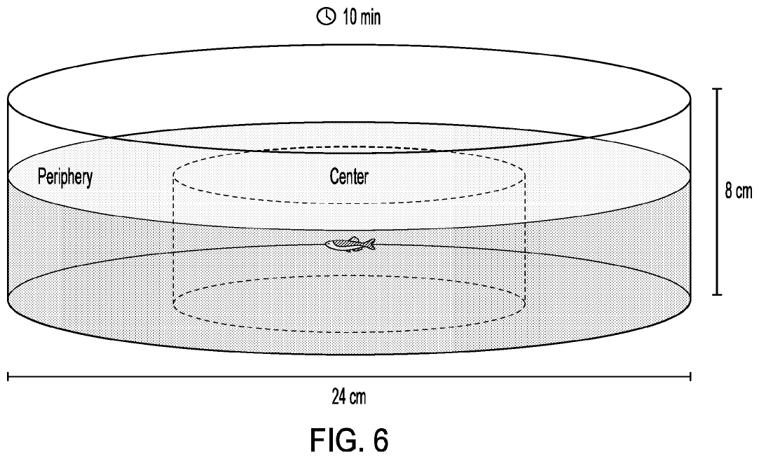

The protocol for the open tank test followed the method described by Benvenutti et al. (2021) Journal of Neuroscience Research 00: 1-16. Immediately after the social interaction test, animals were placed in the center of the open tank arena and recorded for 10 min. The apparatus consisted of a white circular arena (24 cm in diameter and 8 cm in height, 2 cm water level). The apparatus format and the acquisition of videos from the top view allows the evaluation of locomotor parameters related to stereotypic behavior (circular movements and absolute turn angle). The water was changed between every animal. The following outcomes were automatically scored using ANY-Maze software (Stoelting Co., Wood Dale, IL, USA): total distance traveled, absolute turn angle, number of clockwise rotations, and immobility time. See FIG. 6.

### f. Statistical Analyses

The sample size to detect a 0.35 effect size f with 0.9 power and 0.05 alpha was calculated using G*Power (version 3.1) for MacOS; this resulted in n=15 animals per group considering the interaction between the factors. GraphPad Prism 8 (version 8.4.3) for macOS was used to run the statistical analyses and plot the results. No animals were excluded, except for two data points in the open tank test due to a failure in video acquisition. Data were analyzed by two-way ANOVA, with alstonine salt or olanzapine pretreatment (Drug_1) and MK-801 treatment (Drug_2) as the main factors. Bonferroni post hoc test was applied as appropriate. The significant level was set at p<0.05. Data were expressed as mean standard deviation.

### 4. Results

### a. Social Interaction Test

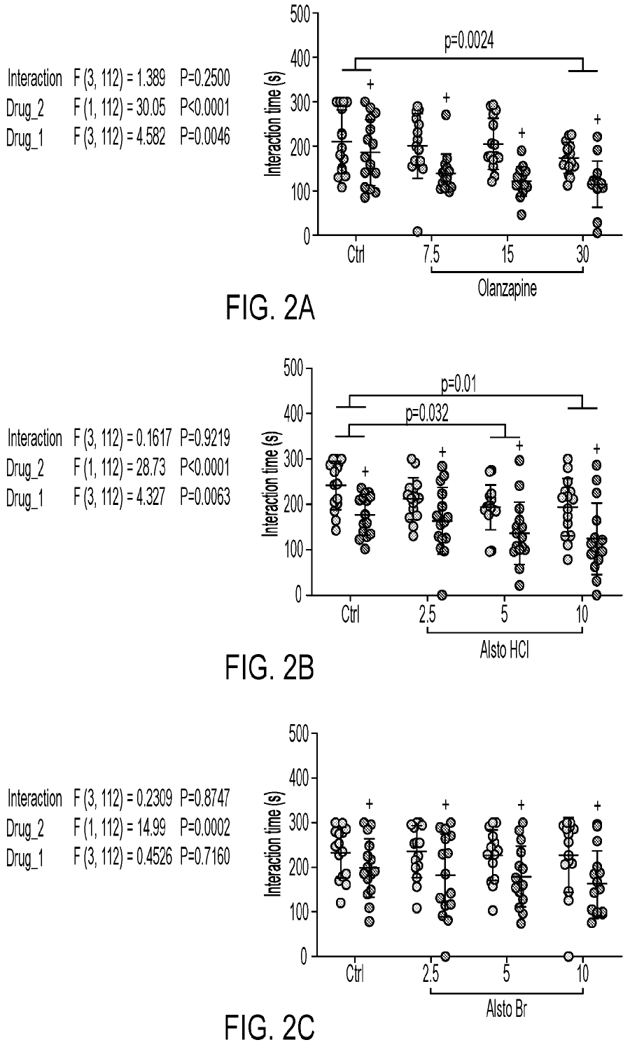

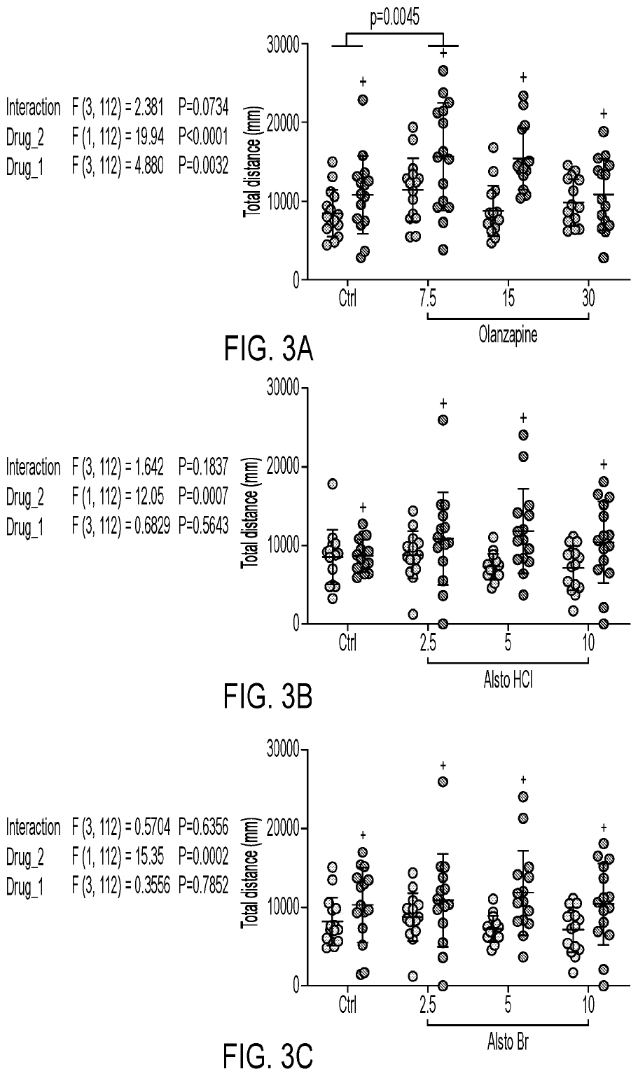

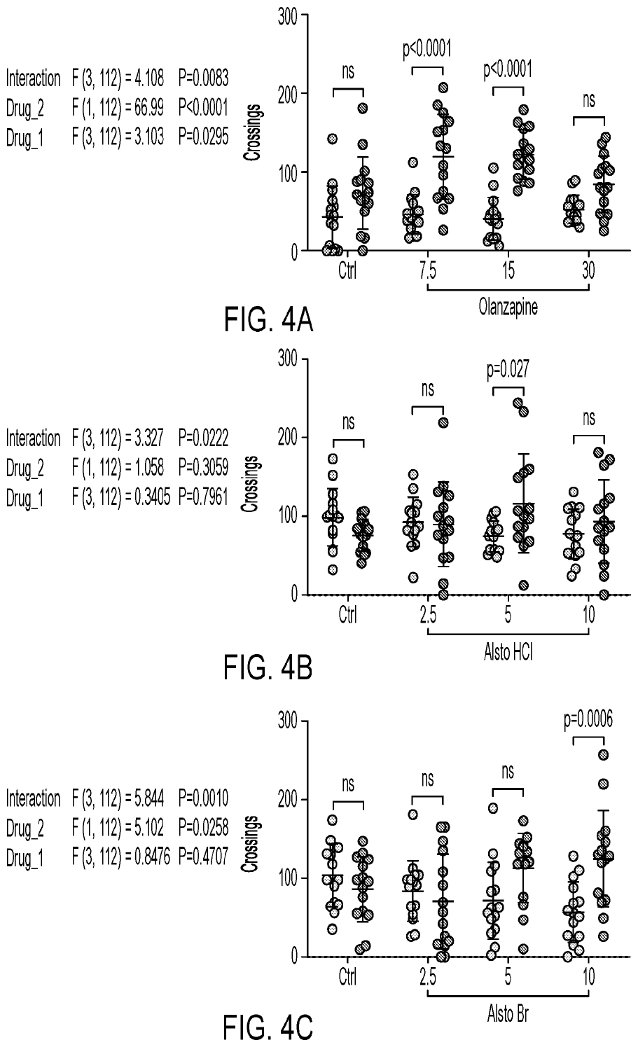

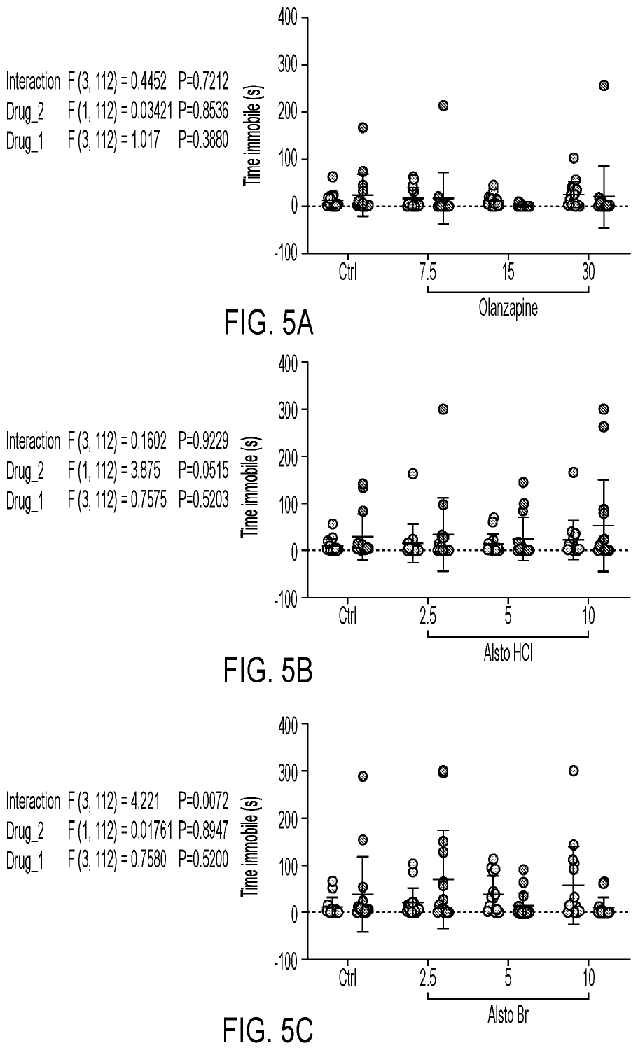

The effects of olanzapine, alstonine hydrochloride, and alstonine hydrobromide in the social interaction test in zebrafish are shown in FIG. 2-5. Time spent in the social stimulus side was measured as a proxy for social interaction (FIG. 2A-C), while total distance traveled (FIG. 3A-C), number of crossings (FIG. 4A-C), and time spent immobile (FIG. 5A-C) were quantified as secondary locomotor parameters. Drug_1: olanzapine, alstonine hydrochloride, or alstonine hydrobromide pretreatment; Drug_2: MK-801. Two-way ANOVA followed by Bonferroni post hoc test; + denotes the main effect of Drug_2 (MK-801). Data are presented as mean±SD. N=15. Ctrl: control, Alsto HCl: alstonine hydrochloride, Alsto Br: alstonine bromide (exposure concentrations are denoted in mg/L).

Referring to FIG. 2A-C, zebrafish exposed to MK-801 spent less time in the side of the tank where conspecifics were presented, denoting decreased social preference in comparison to control groups (Drug_2 main effect: F_{1,112}=30.05, p<0.0001; 28.73, p<0.0001; and 14.99, p=0.0002). Administration of olanzapine did not prevent the MK-801-induced decrease, and had a main effect of decreasing social interaction time at higher concentrations (FIG. 2A; Drug_1 main effect: F_{3,112}=4.582, p=0.0046). Similar effects were observed with alstonine hydrochloride (FIG. 2B; Drug_1 main effect: F_{3,112}=4.327, p=0.0063); Bonferroni post hoc tests revealed that animals exposed to 5 and 10 mg/L alstonine hydrochloride spent less time in the interaction zone as compared to controls. Although administration of alstonine bromide also did not prevent the effects of MK-801, unlike olanzapine and alstonine hydrochloride, none of the concentrations decreased social isolation (FIG. 2C; Drug_1 main effect: F_{3,112}=0.4526, p=0.7160).

Referring to FIG. 3A-C, administration of MK-801 increased the total distance traveled relative to controls (Drug_2 main effect: F_{1,112}=19.94, p<0.0001; 12.05, p=0.0007; and 15.35, p=0.0002). Administration of olanzapine in itself increased total distance traveled in the smallest dose (FIG. 3A; Drug_1 main effect: F_{3,112}=4.880, p=0.032), while administration of either alstonine hydrochloride (FIG. 3B; Drug_1 main effect: F_{3,112}=0.6829, p=0.5643) or alstonine hydrobromide (FIG. 3C; Drug_1 main effect: F_{3,112}=0.3556, p=0.7852) had no such effect.

Referring to FIG. 4A-C, administration of MK-801, in isolation, had no effect on the number of crossings. A significant interaction between MK-801 and either olanzapine (FIG. 4A; Interaction effect: F_{3,112}=4.108 p=0.0083), alstonine hydrochloride (FIG. 4B; Interaction effect: F_{3,112}=3.327, p=0.0222), or alstonine hydrobromide (FIG. 4B; Interaction effect: F_{3,112}=5.844, p=0.0010) was observed. Bonferroni post hoc tests within each Drug_1 level revealed that MK-801 increased the number of crossings only in animals pre-exposed to 7.5 or 15 mg/L of olanzapine, 5 mg/L alstonine hydrochloride, or 10 mg/L of alstonine hydrobromide.

Referring to FIG. 5A-C, MK-801 tended to decrease the time spent immobile (Drug_2 main effect: F_{1,112}=0.03421, p=0.8536; 3.875, p=0.0515; and 0.01761, p=0.8947). A significant interaction between MK-801 and alstonine hydrobromide was observed (FIG. 5C; Interaction effect: F_{3,112}=4.221, p=0.0072); this was driven by alstonine bromide effects in opposite directions in animals exposed or not to MK-801. While MK-801 exposed animals displayed higher mean immobility as compared to their respective controls in groups pretreated with 0 or 2.5 mg/L alstonine bromide, the opposite relationship was observed for the highest alstonine bromide concentrations. Although no statistically significant post hoc comparisons were observed, it seems that alstonine bromide reduced the effects of MK-801 at the highest concentrations tested.

### b. Open Tank Test

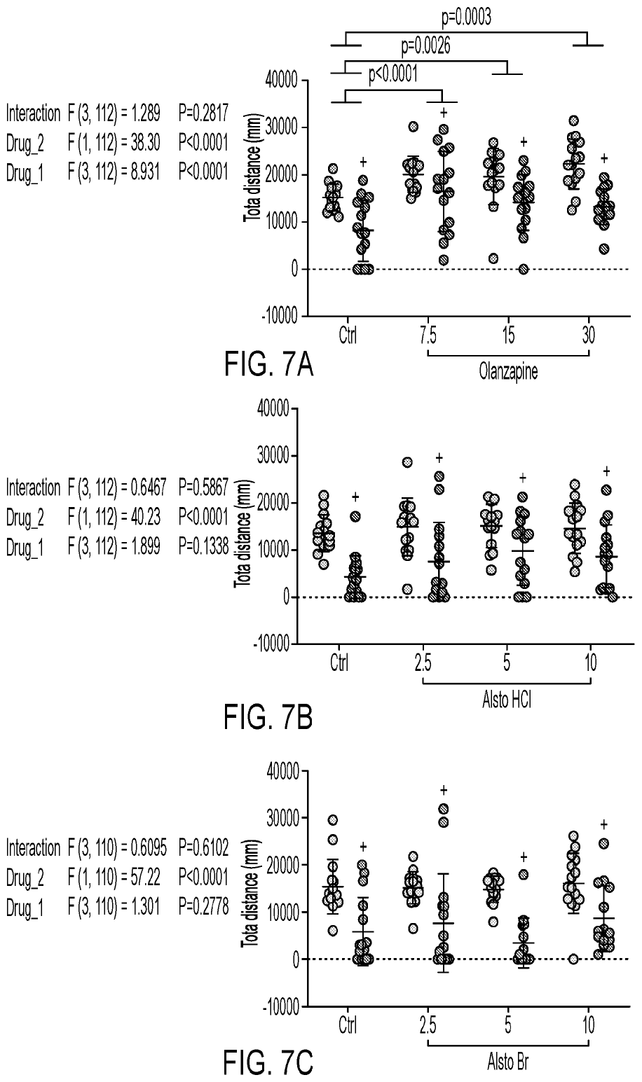

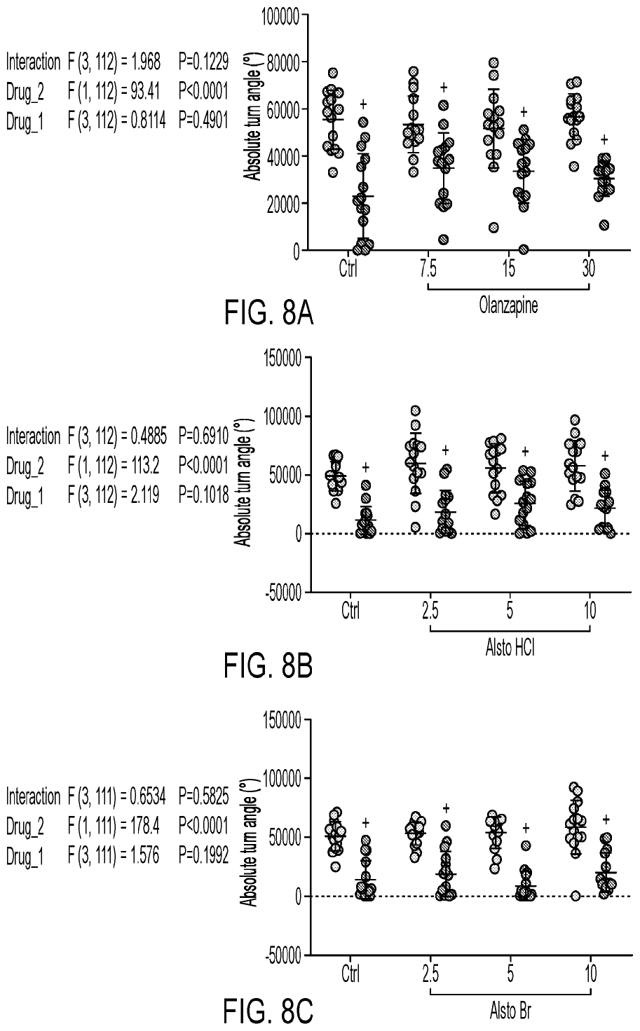

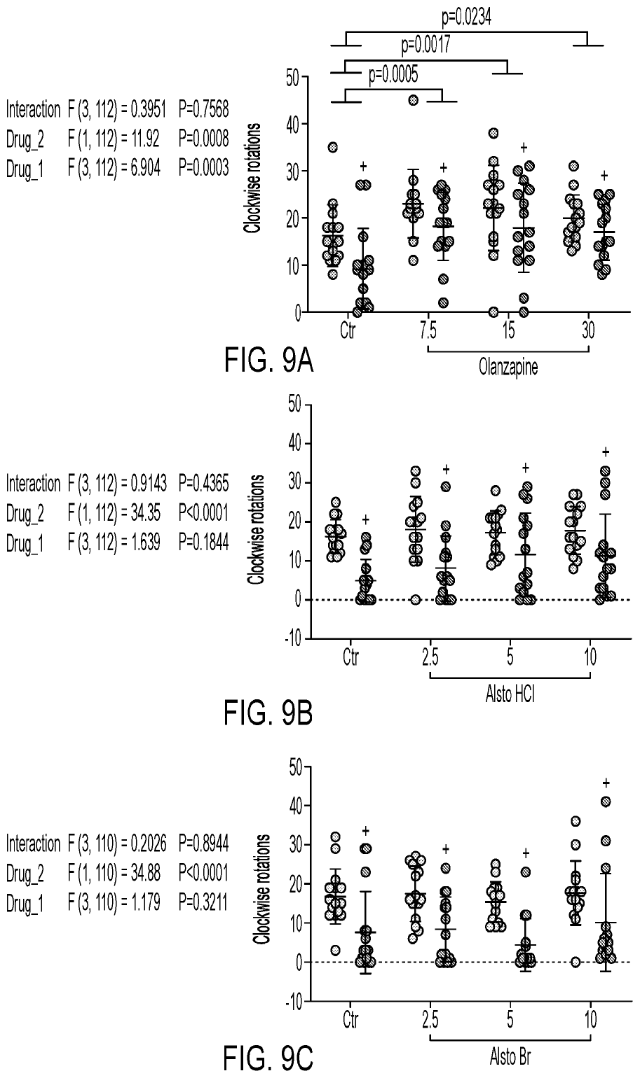

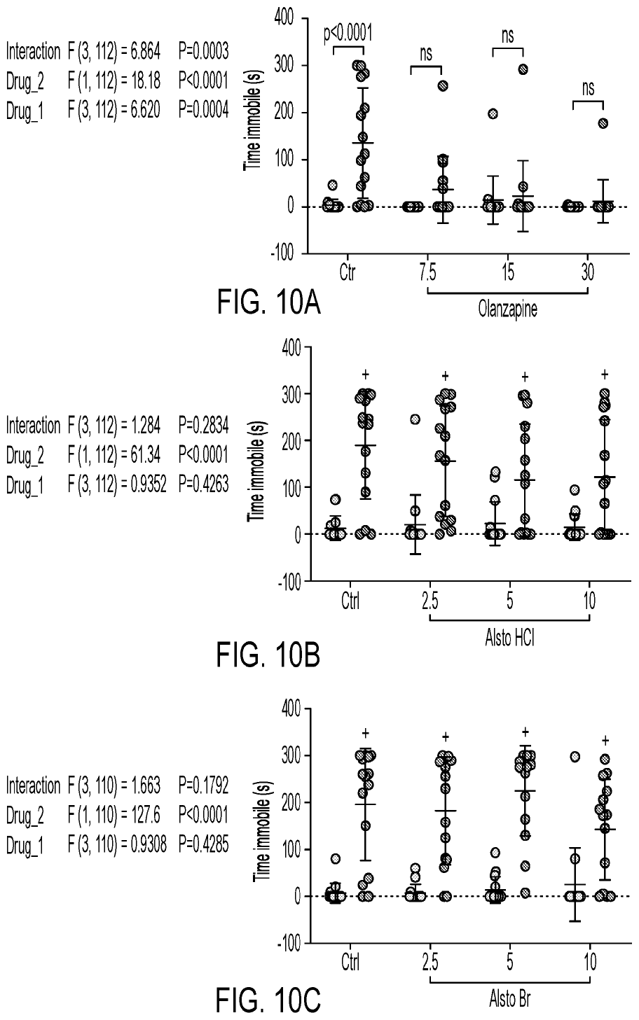

The effects of olanzapine, alstonine hydrochloride, or alstonine hydrobromide in the open tank test in zebrafish are shown in FIG. 7-10. Outcomes evaluated were distance traveled (FIG. 7A-C), absolute turn angle (FIG. 8A-C), clockwise rotations (FIG. 9A-C), and time spent immobile (FIG. 10A-C). Drug_1: olanzapine, alstonine hydrochloride, or alstonine hydrobromide pretreatment; Drug_2: MK-801. Two-way ANOVA; + denotes the main effect of Drug_2 (MK-801). Data are presented as mean±SD. N=13-15. Ctrl: control, Alsto HCl: alstonine hydrochloride, Alsto Br: alstonine bromide (exposure concentrations are denoted in mg/L).

Referring to FIG. 7A-C, MK-801 decreased the total distance relative to controls (Drug_2 main effect: F_{1,112}=38.30, p<0.0001; 40.23, p<0.0001; and 57.22, p<0.0001). Although administration of olanzapine did result in an increase in the total distance (Drug_1 main effect: F_{3,112}=8.931, p<0.0001), there was no interaction with MK-801 (FIG. 7A). Administration of either alstonine hydrochloride (FIG. 7B) or alstonine hydrobromide (FIG. 7C) did not have an effect, as no main effects for Drug_1 or interaction effects were observed.

Referring to FIG. 8A-C, administration of MK-801 decreased the absolute turn angle relative to controls (Drug_2 main effect: F_{1,112}=93.41, p<0.0001; 113.2, p<0.0001; and 178.4, p<0.0001). Neither administration of olanzapine (FIG. 8A), alstonine hydrochloride (FIG. 8B), or alstonine hydrobromide (FIG. 8C) had an effect, as no main effects for Drug_1 or interaction effects were observed.

Referring to FIG. 9A-C, administration of MK-801 decreased the number of clockwise rotations relative to controls (Drug_2 main effect: F_{1,112}=11.92, p=0.0008; 34.35, p<0.0001; and 34.88, p<0.0001). Although administration of olanzapine did result in an increase in the number of clockwise rotations (Drug_1 main effect: F_{3,112}=6.904, p=0.0003), there was no interaction with MK-801 (FIG. 9A; Interaction effect: F_{3,112}=0.3951, p=0.7568). Administration of either alstonine hydrochloride (FIG. 9B) or alstonine hydrobromide (FIG. 9C) did not have a main effect or an interaction effect.

Referring to FIG. 10A-C, MK-801 increased the time spent immobile (Drug_2 main effect: F_{1,112}=18.18, p<0.0001; 61.34, p<0.0001; and 127.6, p<0.0001). Administration of olanzapine at either 7.5, 15, or 30 mg/L prevented this effect (FIG. 10A; Interaction effect: F_{3,112}=6.864, p=0.0003). Although administration of either alstonine hydrochloride (FIG. 10B) or alstonine hydrobromide (FIG. 10C) did not have an effect (main or interaction), the data became more dispersed as the concentration increased. Without wishing to be bound by theory, this indicates that higher doses of the alstonine salts can be effective.

### 5. Discussion

A summary of the results obtained in the social interaction and the open tank tests are shown in Table 3.

TABLE 3

| | | MK- | | | |
|---|---|---|---|---|---|
| | Outcome | 801 | Olanzapine | Alsto HCl | Alsto Br |
| Social | Interaction | ↓ | ↓ (main | ↓ (main | N.E.¹ |
| Inter- | time | | effect, 30) | effect, 5/10) | |
| action | Total | ↑ | ↑ (main | N.E.¹ | N.E.¹ |
| Test | distance | | effect, 7.5) | | |
| | Crossings | = | ↑ (Ctrl vs. | ↑ (Ctrl vs. | ↑ (Ctrl vs. |
| | | | MK, 7.5/15) | MK, 5) | MK, 10) |
| | Time | = | N.E.¹ | N.E.¹ | Interaction |
| | immobile | | | | |
| Open | Total | ↓ | ↑ (main | N.E.¹ | N.E.¹ |
| Tank | distance | | effect. 7.5/ | | |
| Test | | | 15/30) | | |
| | Absolute | ↓ | N.E.¹ | N.E.¹ | N.E.¹ |
| | turn angle | | | | |
| | Clockwise | ↓ | ↑ (main | N.E.¹ | N.E.¹ |
| | rotations | | effect. 7.5/ | | |
| | | | 15/30) | | |
| | Time | ↑ | Prevents | N.E.¹ | N.E.¹ |
| | immobile | | MK effect | | |

¹N.E. = no main or interaction effect observed at the concentrations tested

NMDA antagonism is a widely used pharmacological tool to model schizophrenia-like phenotypes in model animals, and recent publications have endorsed the use of zebrafish as an alternative model animal to study schizophrenia and screen for potential novel treatments (Bruni et al. (2016) Nat Chem Biol 12: 559-566; Gawel et al. (2019) Neurosci Biobehav Rev 107: 6-22). Here, MK-801 was used to acutely induce behavioral alterations of translational relevance to schizophrenia in zebrafish, and ultimately evaluate the preventative effects of alstonine salts against the deficits caused by NMDA antagonism.

As expected, a reduced time was observed in the social side as well as hyperlocomotion in groups exposed to MK-801 in the social interaction test. On the other hand, MK-801 decreased the distance traveled in the open tank test. Such divergent effects on locomotion are in line with previous reports on the context-dependent effects of NMDA antagonists in zebrafish (Benvenutti et al. (2021) J Neurosci Res 99: 2844-2859; Tran et al. (2016) Behav Brain Res 296: 26-29). A decrease in absolute turn angle, clockwise rotations, and immobility time was also observed in groups exposed to MK-801 in the open tank test, as expected.

Olanzapine failed to demonstrate a clear ability to reverse the schizophrenia-like symptoms produced by MK-801 in most of the parameters tested. Such data appears to correspond with that previously obtained in established mice models. See, e.g., Morimoto et al. (2002) Neuropsychopharmacology 26(4): 456-67 and Menezes, C. (2010) “Effects of typical and atypical antipsychotics on the MK801-induced social interaction” (Dissertion, TCC Pharmacy School). The alstonine salts evaluated similarly failed to reverse the MK-801 induced symptoms, although the varied effects observed upon administration of either salt in isolation suggests that they may have a different activity profile than olanzapine and be less prone to induce adverse effects. Further, the observation of a dispersion amongst the data points in certain parameters with increasing doses suggests that higher concentrations of the salts, and, in particular, the bromide salt, may be advantageous.

Data in the literature are quite contradictory regarding the potential of antipsychotics to prevent social interaction deficits caused by glutamatergic antagonists. Sulpiride, for example, acutely prevents the deficits caused by MK-801 (de Moura Linck in 2008 (Prog Neuropsychopharmacol Biol Psychiatry 32(6): 1449-52), but olanzapine when administered acutely does not show a similar profile (Morimoto et al. (2002) Neuropsychopharmacology 26(4): 456-67). Data for risperidone (Corbett et al. (1993) Pharmacol Biochem Behav 45(1): 9-17; Morimoto et al. (2002) Neuropsychopharmacology 26(4): 456-67; Becker and Grecksch (2004) Prog Neuropsychopharmacol Biol Psychiatry 28(8): 1267-77) and clozapine (de Moura Linck in 2008 (Prog Neuropsychopharmacol Biol Psychiatry 32(6): 1449-52; Sams-Dodd (1996) Behav Pharmacol 7(1); 3-23) also yield apparently contradictory results. The data in rodents replicate the data in humans, since none of the antipsychotics agents in the market (typical or atypical) are effective against negative symptoms in patients with schizophrenia (Lieberman et al. (2005) N Engl J Med 353: 1209-1223.

In sum, the results herein demonstrate that alstonine hydrochloride and alstonine hydrobromide behave in a manner consistent with previous findings with antipsychotic agents in mice models. Moreover, the distinct behavior of alstonine bromide compared to alstonine chloride suggests that advantages may be obtained via the bromide salt. For example, in the social interaction test, administration of alstonine hydrochloride at 5 or 10 mg decreased the interaction time, whereas administration of alstonine bromide did not. In addition, the data points of the hydrobromide salt become more dispersed as the concentration increases (i.e., at 10 mg versus 5 mg), suggesting that a reversal of the MK-801-induced effects may yet be possible at higher doses.

### M. Prophetic Examples

### 1. Models to Demonstrate Utility in Treating Psychotic Disorders

Protocols for evaluating utility in treating psychotic disorders are well-known in the art. See, e.g., Costa-Campos et al. (1998) Pharmacology Biochemistry and Behavior 60(1): 133-141; WO 2020/072675 A1; Linck et al. (2011) Evidence-Based Complementary and Alternative Medicine, Volume 2011, Article ID 418596, 7 pages; Linck et al. (2008) Progress in Neuro-Psychopharmacology & Biological Psychiatry 32: 1449-1452; Herrmann et al. (2012) Neurochemistry International 61: 1144-1150; Linck et al. (2014) Phytomedicine 22(1): 52-5. Exemplary protocols are detailed more fully below.

Mouse models will be used to demonstrate the potential of the disclosed compounds in treating and or affecting psychotic disorders including cognitive impairment of schizophrenia, negative symptoms of schizophrenia including deficit in social interaction, bipolar disorder, major depression including psychotic major depression, PCP-induced hyperlocomotion suppression for positive (psychotic) symptoms of schizophrenia, psychoses of Alzheimer's disease and Parkinson's disease, obsessive compulsive disorder, Tourette's syndrome, and age-associated cognitive impairment.

Locomotor Activity (LMA). The ability of a disclosed compound to block the increase in Locomotor Activity (LMA) produced by amphetamine or the glutamate receptor agonist, phencyclidine (PCP), has a high predictive value for the antipsychotic efficacy of the disclosed compound for treating schizophrenia. Administration of amphetamine or PCP to man has been shown to produce a psychotic state in a significant percentage of previously normal humans. Treatment of mice with PCP for one week has been shown to produce a robust model of schizophrenia. As such, the ability of the disclosed compounds to reduce LMA in mice treated with amphetamine or PCP will be evaluated.

Novel Object Recognition Test. The Novel Object Recognition test in rodents is considered a valid model of spatial memory in man. The time spent viewing an object seen previously versus the time spent viewing a novel object (i.e., the Discrimination Index (DI)) provides an indirect measure of assessing retention of the memory of the object that had previously been shown to the rodent. Normal mice remember for ˜24 hours. The time to loss of memory in aged mice can be as low as zero to less than 8 hours. As such, the ability of the disclosed compounds to reduce DI in mice treated with PCP will be evaluated.

A healthy mouse will vigorously explore a newly introduced mouse and retain the memory for that mouse for up to 24 hours. The loss of interest in exploring a novel mouse is considered a sign of negative symptoms. PCP-treatment produces a deficit in social interaction, which models negative symptoms in schizophrenia. These include lack of motivation, loss of interest in activities, diminished capacity for pleasure, and spontaneous activity. As such, the effects of the disclosed compounds on social interaction in mice treated with PCP, including evaluation of social behaviors such as sniffing, following, and avoiding Object Exploration via interactions with inanimate objects, will be evaluated.

Porsolt Forced Swim Test. The Porsolt Forced Swim Test (FST) is a gold standard test for predicting antidepressant action. In the FST, normal mice become immobile after being required to swim in a tank, and begin to float instead of struggling to get out of the water. Antidepressant drugs have been shown to increase the amount of time that a mouse will continue to swim prior to floating. As such, the ability of the disclosed compounds to decrease immobility time in the FST will be evaluated.

Marble Burying. Marble Burying (MB) and extent of shredding a standard type of rodent bedding (i.e., Nestlet Shredding) are both proven methods for assessing drug efficacy to treat obsessive-compulsive disorder and Tourette's syndrome. As such, the effects of the disclosed compounds on Marble Burying and Nestlet Shredding will be evaluated.

Memory. Aged mice show a decline in memory function beginning around 13-15 month of age. Drugs that enhance memory can be assessed using the Novel Object Recognition test and determination of the Discrimination Index (DI) as detailed above. As such, the ability of the disclosed compounds to improve DI for aged mice (e.g., 22-month old mice) versus young mice (e.g., 2.5-month old mice) will be evaluated.

### 2. Models to Demonstrate Utility in Treating Anxiety Disorders

Protocols for evaluating utility in treating anxiety disorders are well-known in the art. See, e.g., Linck et al. (2012) Progress in Neuro-Psychopharmacology & Biological Psychiatry 26: 29-33; Costa-Campos et al. (2004) Pharmacology, Biochemistry and Behavior 77: 481-489. Exemplary protocols are detailed more fully below.

### a. Working Memory Deficity

Step-down inhibitory avoidance. The protocol is adapted from Barros et al. (2005) Neurobiol Learn Mem 83: 113-8. Mice will be habituated to the dimly lit experimentation room for at least 30 min before the procedure. The inhibitory avoidance training apparatus is, for example, a plastic box of ˜30×30×40 cm, with a fixed platform (˜5×5×4 cm) at the center of the grid floor. Mice will be individually placed on the platform, and the latency to step-down (four paws on the grid) will be automatically recorded in training and test sessions. In the training session, upon stepping a down, the mouse will receive a 0.3 mA scrambled foot shock for 5 s. Test sessions will be performed 10 s later, with the same procedure except that no shock will be administered after stepping down; a 300-s cut-off time will be set for stepping down.

Working memory assessment. Mice (n=14-17) will be treated with saline, clozapine (2 mg/kg), sulpiride (10 mg/kg), or a disclosed compound (0.5 or 1 mg/kg). Thirty minutes after treatment mice will be subjected to the training session.

MK801-induced working memory deficit Mice (n=23-31) will be likewise treated with saline, clozapine, sulpiride, or a disclosed compound; 30 min later mice will receive a second treatment with either saline or MK801 (0.05 mg/kg). The step-down training session will be performed 30 min after the last treatment.

### b. Dependence on 5-HT_{2A/C} Receptors

In order to evaluate the involvement of 5-HT_{2A}/c on the antipsychotic-like effects of the disclosed compounds, mice will be pre-treated with the 5-HT_{2A/C} receptor antagonist ritanserin, altanserine, and/or SB242084 before behavioral tests. Ritanserine doses and administration schedules will be based on Su et al. (2007) Eur J Pharmacology 564: 123-30, as well as on previous studies showing that ritanserin is devoid of effects per se. See, e.g., Linck et al. (2012) Progress in Neuro-Psychopharmacology & Biological Psychiatry 26: 29-33. Altanserine will be adminstered at 0.01 mg/kg and SB242084 at 0.5 mg/kg.

MK801-induced working memory deficit After habituation mice (n=9-17) will receive saline or the 5-HT_{2A/C} antagonist ritanserin (0.1 mg/kg); 10 min later animals will be treated with saline or a disclosed compound 1 mg/kg, followed 30 min later by a third administration of either saline or MK801 (0.05 mg/kg). Working memory will be tested as described above, 30 min after the last treatment.

MK801-induced social withdrawal. Method is adapted from Rung et al. (2005) Biol Psychiatry 29: 827-32. Mice will be acclimatized to a reversed 12-h light cycle (lights on at 20:00 h), housed at 8/cage (familiar group) for 2 weeks before the experiments. Mice will be randomly assigned to groups (n=8-13 pairs) that received saline or ritanserin (0.1 mg/kg), and 10 min later will be treated with saline or a disclosed compound 1 mg/kg. Social withdrawal will be induced with MK801 (0.3 mg/kg), given 30 min after the second saline or ritanserin treatment (Rung et al. (2005) Biol Psychiatry 29: 827-32). Experiments will be performed 30 min after the last treatment, in a faintly lit room (e.g., red bulb, 40 W); the social interaction apparatus (test box) can consist of, for example, a topless transparent acrylic box (˜25×20×20 cm). Forty-eight and 24 h before the test, mice will be individually submitted to 10 min habituation sessions in the test box. On the test day, mice will be allocated to selected pairs so that the two animals come from unfamiliar groups (different home cages), have matching body weights, and receive the same drug treatment. The behavior of each pair will be video-recorded in the test box for 10 min; the time spent in social interaction (sniffing and grooming the partner, following, mounting, and crawling under or over the partner) will be analyzed later by a trained observer, blind to experimental groups, using software such as, for example, The Observer® XT5.0 (Noldus Information Technology, Wageningen, The Netherlands). Passive contact (sitting or lying with bodies in contact) will not be considered as social interaction.

MK801-induced hyperlocomotion. The method is adapted from Ninan and Kulkami (1998) Eur J Pharmacol 358: 111-6. Activity cages (e.g., 45×25×20 cm) will be equipped with three parallel photocells, which automatically record the number of crossings. Mice (n=6-9) will be treated with saline or ritanserin (0.1 mg/kg), and 10 min later with saline or a disclosed compound 1 mg/kg; 30 min after the second treatment the animals will receive saline or MK801 (0.25 mg/kg). Mice will be individually placed in the activity cages 30 min after the last administration, and locomotion will be recorded from the 5th minute for 10 min (first 5 min considered as exploratory behavior).

### 3. Models to Demonstrate Utility in Treating Epilepsy

Protocols for evaluating utility in treating epilepsy are well-known in the art. See, e.g., Costa-Campos et al. (2004) Journal of Ethnopharmacology 93: 307-310; Löscher and Schmidt (2011) Epilepsia 52: 657-678; Löscher (2011) Seizure 20: 359-368; Swinyard and Kupferberg (1985) Fed. Proc. 44: 2629-2633; Gaikwad et al. (2021) Journal of Drug Delivery and Therapeutics 11(2-s): 175-178. Exemplary protocols are detailed more fully below.

Maximal Electroshock (MES) Induced Convulsions. The animals will be divided into five groups, each group comprising, for example, six rats. Different groups will be treated with distilled water (10 mL/kg), diazepam (5 mg/kg), disclosed compound, and ScMeOH at doses of 125, 250, and 500 mg/kg, BW. Thirty minutes later, convulsions will be induced in all the groups of animals using electroconvulsiometer. A 60 Hz alternating current of 150 mA for 2 s will be delivered through the ear electrodes. The animal will be observed for the occurrence of tonic hind limb extension. As such, the ability of the disclosed compounds to protect animals from MES (i.e., tonic extension of the hind limbs) will be evaluated.

Pentylenetetrazol-(PTZ-) induced convulsions in mice. Swiss albino mice of 4-6 weeks of either sex weighing to 20-30 g will be randomly selected and marked to permit individual identification, and further grouped into four groups each group comprising 6 animals. Control: Distilled water (5 ml/kg, p.o.)+PTZ (80 mg/kg, i.p.) Standard: Diazepam (4 mg/kg, i.p.)+PTZ (80 mg/kg, i.p.) Test I: Ethanolic leaves extract (100 mg/kg, p.o.)+PTZ (80 mg/kg, i.p.) Test II: Ethanolic leaves extract (200 mg/kg, p.o.)+PTZ (80 mg/kg, i.p.). The disclosed compound will be administered continuously for a period of 7 days. On the 7th day, convulsions will be induced by PTZ. As such, the ability of the disclosed compounds to protect animals from PTZ will be evaluated.

Picrotoxin-induced convulsions. Groups of 10 mice of either sex with a weight between 18 and 22 g will be treated either orally or i.p. with the disclosed compounds or the standard (e.g., 10 mg/kg diazepam i.p.). Thirty min after i.p. treatment or 60 min after oral administration the animals will be injected with 3.5 mg/kg s.c. picrotoxin and will be observed for the following symptoms during the next 30 min: clonic seizures, tonic seizures, death (Wolfgang et al., Drug Discovery and Evaluation, Pharmacological Assays, second Edition).

Kainic acid (KA) model. Systemic administration of the appropriate dose of KA induces “wet dog shakes,” generalized tonic-clonic convulsions, teeth chattering, and altered motor activity, including an initial hypo-activity, which transforms to a hyperactivity at later stage. Neurodegeneration occurs in the pyramidal layer of CA3 area of hippocampus and in the piriform cortex as early as 3 hours following injection. At this time point, a positive correlation exists between the dose of KA and the extent of the acute neurochemical changes including increases of 3, 4-dihydroxyphenylacetic acid and decrease in noradrenaline levels in all brain regions investigated. By 13 hours to 2 weeks, neuronal somata degenerate and disappear in areas such as the olfactory cortex and parts of the amygdaloid complex, hippocampal formation, thalamus and neocortex (Kasthuri and Kavimani (2013) International Journal of Pharmaceutical and Applied Sciences 18).

Bicuculline model. Bicuculline has been applied focally and systemically. It has been used to induce acute simple focal epilepsy after topical application in the sensorimeter cortex in rats. Another model using biculline with induction of chronic simple partial seizures was developed by Remler and co-worker. This model mixes features of focal and generalized epilepsy and is referred to as systemic focal epileptogenesis. In this model, rats receive radiation to a limited volume (0.25 ml) of cerebrum. Three to six months later, when the blood-brain barrier is injected systemically, inducing an epileptic focus with recurrent EEG spikes and focal seizures enduring for several weeks after a single injection. The spikes are suppressed by phenytoin, Phenobarbital, chlorodiazepoxide, and valproic acid (Fisher et al. (2005) Epilepsia 46(4): 470-2). Bicuculline is believed to exert its epileptogenic effect through blocking GABA ergic neurotransmission by competing with GABA for its binding sites (Kasthuri and Kavimani (2013) International Journal of Pharmaceutical and Applied Sciences 18).

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A compound having a structure represented by a formula: 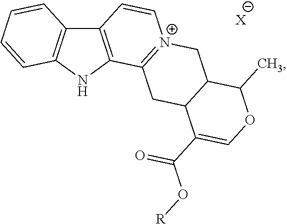
wherein X is selected from halogen, acetate, alginate, ascorbate, benzoate, carbonate, cinnamate, citrate, diphosphate, fumarate, gluconate, lactate, laurate, malate, maleate, mesylate, myristate, nitrate, palmitate, phosphate, propionate, sorbate, succinate, sulfate, tartrate, and a conjugate base of a phenolic acid; and
wherein R is C1-C16 alkyl,
provided that when R is methyl then X is not Cl.

2. The compound of claim 1, wherein X is halogen.

3. The compound of claim 1, wherein X is selected from F, Br, and I.

4. The compound of claim 1, wherein X is selected from acetate, alginate, ascorbate, benzoate, carbonate, cinnamate, citrate, diphosphate, fumarate, gluconate, lactate, laurate, malate, maleate, mesylate, myristate, nitrate, palmitate, phosphate, propionate, sorbate, succinate, sulfate, tartrate, and a conjugate base of a phenolic acid.

5. The compound of claim 4, wherein X is substituted with 0-1, 0-2, or 0-1, 0-2, or 0-3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl.

6. The compound of claim 1, wherein X is the conjugate base of a phenolic acid.

7. The compound of claim 6, wherein the phenolic acid is salicylate acid, p-hydroxybenzoic acid, p-coimaric acid, ferulic acid, syringic acid, sinapic acid, vanillic acid, caffeic acid, or 3,4-dihydroxybenzoic acid.

8. (canceled)

9. The compound of claim 1, wherein X is selected from cinnamate, succinate, and trimethoxybenzoate.

10. The compound of claim 1, wherein R is methyl.

11. The compound of claim 1, wherein R is C2-C16 alkyl.

12. (canceled)

13. The compound of claim 1, having a structure represented by a formula:

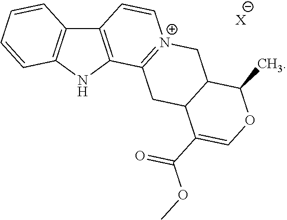

14. (canceled)

15. The compound of claim 1, having a structure represented by a formula:

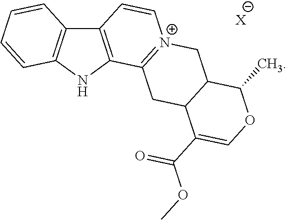

16. (canceled)

17. The compound of claim 1, having a structure represented by a formula:

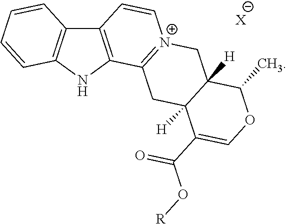

18-19. (canceled)

20. The compound of claim 1, having a structure represented by a formula:

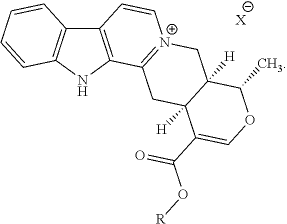

21. The compound of claim Error! Reference source not found, wherein X is selected from cinnamate, succinate, and trimethoxybenzoate.

22. (canceled)

23. The compound of claim 1, wherein the compound is selected from:

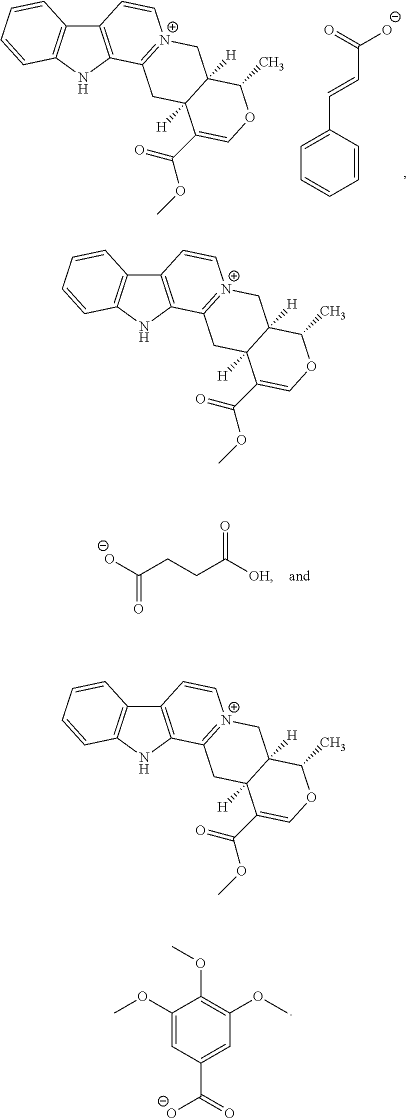

24. A pharmaceutical composition comprising an effective amount of the compound of claim 1, and a pharmaceutically acceptable carrier.

25-58. (canceled)

59. A method for treating a disorder in a subject in need thereof, the method comprising administering to the subject an effective amount of the compound of claim 1, wherein the disorder is a psychotic disorder, an anxiety disorder, or epilepsy.

60-65. (canceled)

66. The method of claim 0, wherein administering is via transdermal or intradermal administration.

67. The method of claim 0, wherein administering is via a transdermal patch or a microneedle array.

68-120. (canceled)
